(19) United States
(12) Patent Application Publication
(10) Pub. No.: US 2026/0301950 A1
(43) Pub. Date: Oct. 1, 2026

(54) CODON 72 OF TP53 SINGLE NUCLEOTIDE POLYMORPHISM AS A PREDICTIVE DISEASE BIOMARKER

(71) Applicant: Texas Tech University System, Lubbock, TX (US)

(72) Inventors: Maciej Markiewski, Abilene, TX (US); Magdalena Karbowniczek, Abilene, TX (US); Ashok Silwal, Abilene, TX (US)

(73) Assignee: Texas Tech University System, Lubbock, TX (US)

(21) Appl. No.: 19/477,334

(22) PCT Filed: Apr. 21, 2024
(86) PCT No.: PCT/US2024/025613

§ 371 Date: Oct. 21, 2025

Related U.S. Application Data
(60) Provisional application No. 63/460,993, filed on Apr. 21, 2023.

(51) Int. Cl. G16H 50/20; C12Q 1/6883; G16B 20/20; G16H 20/10; G16H 50/70

(52) U.S. Cl. CPC G16H 50/20; C12Q 1/6883; G16B 20/20; G16H 20/10; G16H 50/70; C12Q 2600/106; C12Q 2600/156

(57) ABSTRACT

Embodiments of the present disclosure pertain to a method of assessing a disease in a subject by receiving a biological sample of the subject; detecting a single nucleotide polymorphism at codon 72 of the P53 protein (SNP) from the biological sample; and correlating the detected SNP to disease prognosis in the subject. Further embodiments pertain to methods of treating a subject by detecting an SNP from a biological sample of the subject and editing the detected SNP to treat or prevent a disease in the subject. Additional embodiments pertain to diagnostic tests and computing devices for assessing a disease in a subject in accordance with the methods of the present disclosure.

### CROSS-REFERENCE TO RELATED APPLICATIONS

This application claims priority to U.S. Provisional Patent Application No. 63/460,993, filed on Apr. 21, 2023. The entirety of the aforementioned application is incorporated herein by reference.

### STATEMENT REGARDING FEDERALLY SPONSORED RESEARCH

This invention was made with government support under Grant No. R01CA190209 awarded by the National Institutes of Health. The government has certain rights in this invention.

### BACKGROUND

Current methods and systems for assessing disease prognosis have numerous limitations. Embodiments of the present disclosure aim to address the aforementioned limitations.

### SUMMARY

In some embodiments, the present disclosure pertains to a method of assessing a disease in a subject. In some embodiments, the methods of the present disclosure include: (1) receiving a biological sample of the subject; (2) detecting a single nucleotide polymorphism at codon 72 of the P53 protein (SNP) from the biological sample; and (3) correlating the detected SNP to disease prognosis in the subject. In some embodiments, the methods of the present disclosure also include a step of implementing a treatment decision based on the prognosis. For instance, in some embodiments, the treatment decision includes, without limitation, editing the detected SNP, monitoring the course of the disease, implementing a disease prevention regimen, implementing a disease treatment regimen, implementing a disease management regimen, or combinations thereof.

Further embodiments of the present disclosure pertain to methods of treating a subject. In some embodiments, the treatment methods of the present disclosure include: (1) detecting a single nucleotide polymorphism (SNP) at codon 72 of the P53 protein from a biological sample of the subject; and (2) editing the detected SNP. In some embodiments, the methods of the present disclosure may be utilized to treat or prevent a disease in a subject.

Additional embodiments of the present disclosure pertain to a diagnostic test for use in assessing a disease in a subject. In some embodiments, the diagnostic test includes: (1) instructions for receiving at least one detected single nucleotide polymorphism at codon 72 of the P53 protein (SNP) from a biological sample of the subject; and (2) instructions for correlating the detected SNP to disease prognosis in the subject. In some embodiments, the diagnostic test also includes instructions for implementing a treatment decision based on the prognosis.

Further embodiments of the present disclosure pertain to a computing device for assessing a disease in a subject. In some embodiments, the computing device includes one or more computer readable storage mediums having a program code embodied therewith. In some embodiments, the program code includes: (1) programming instructions for receiving at least one detected single nucleotide polymorphism at codon 72 of the P53 protein (SNP) from a biological sample of the subject; and (2) programming instructions for correlating the detected SNP to disease prognosis in the subject. In some embodiments, the computing devices of the present disclosure also include programming instructions for recommending a treatment decision based on the prognosis.

### DRAWINGS

FIG. 1A illustrates a method of assessing a disease in a subject in accordance with various embodiments of the present disclosure.

FIG. 1B illustrates a method of treating a subject in accordance with various embodiments of the present disclosure.

FIG. 1C illustrates a computing device for assessing a disease in a subject in accordance with various embodiments of the present disclosure.

FIGS. 2A-2U show biomarkers of inflammation in COVID-19 patients, including total monocytes count (FIG. 2A); C-reactive protein (CRP) level (FIG. 2B); and inflammatory cytokines and chemokines measured based on Quantibody array (FIGS. 2C-2U). Only homozygous patients were analyzed. Data points represent mean±SEM (N=25-42). *P<0.05, **P<0.01, ***P<0.001, and ****P<0.0001 by t-test.

FIGS. 3A-3M show macrophage activation markers. FIGS. 3A-3J show expression of genes associated with M1 (FIGS. 3A-3D) and M2 phenotypes (FIGS. 3F-3J) in murine macrophages stimulated with LPS and IL-4, respectively, by q RT-PCR. Data were normalized for GAPDH. FIG. 3E shows IL-12 in macrophage supernatant after LPS stimulation by ELISA. Data points represent mean±SEM from three independent experiments. FIGS. 3K-3M show M1 genes in human macrophages (n=5-6). *P<0.05, **P<0.01, ***P<0.001, and ****P<0.0001 by one-way ANOVA.

FIGS. 4A1-4G show a PI3K/Akt pathway and its downstream targets. Shown are western blotting of Akt expression (total [t]) and phosphorylation (p) based on whole cell lysates (WCL) (FIGS. 4A1-4A2); mTORC1 activation based on WCL (FIGS. 4B1-4B3); t-FOXO3a and p-FOXO3a based on WCL (FIGS. 4C1-4C2); Cytoplasmic p-FOXO3a (FIGS. 4D1-4D2); Nuclear t-FOXO3a (FIGS. 4E1-4E2); Nuclear p-MDM2 (FIGS. 4F1-4F2); and t-MDM2 based on WCL (FIG. 4G). Bar graphs show means±SEM. Data are representative of at least two independent experiments.

FIGS. 5A-5F2 show phospho-FOXO3a-NF-kB RelA interaction and p53 nuclear export. FIG. 5A shows FOXO3a and NF-κB RelA detection by western blotting in FOXO3a immunoprecipitation (IP) from whole cell lysate (WCL) of unstimulated or LPS-stimulated macrophages (ellipse-FOXO3a-NFκB interaction). FIG. 5B1 shows NF-κB RelA and FOXO3a by immunofluorescence (DAPI-blue nuclear staining, arrows indicate colocalization, scale bar-30 μm). FIGS. 5B2 and 5B3 show quantification of data from FIG. 5B1. FIG. 5C1 show nuclear NF-κB RelA by western blotting. FIG. 5C2 shows densitometry based on FIG. 5C1. FIG. 5D1 shows cytoplasmic NF-κB RelA by western blotting. FIG. 5D2 shows densitometry based on FIG. 5D1. FIG. 5E1 shows nuclear phosphor (p)-p53 by western blotting. FIG. 5E2 shows densitometry based on FIG. 5E1. FIG. 5F1 shows p53 by immunofluorescence (DAPI-blue nuclei, scale bar-50 μm). FIG. 5F2 shows nuclear staining quantification based on FIG. 5F1. Horizontal lines (FIGS. 5B2, 5B3 and 5F2) and bar graphs (FIG. 5E2) represent Mean±SEM. *p<0.05, **p<0.01; ***p<0.001, and ****p<0.0001 by t-test. Data are representative of two-three independent experiments.

FIGS. 6A1-6D2 show the impact of Akt inhibition on R72 macrophages phenotype. FIG. 6A1 shows Phospho (p)-Akt and p-FOXO3a based on whole cell lysates (WCL) and nuclear NF-κB RelA from unstimulated and LPS stimulated R72 macrophages without or preincubated with Akt inhibitor MK-2206 by western blotting. FIGS. 6A2 and 6A3 show densitometry based on FIG. 6A1. FIG. 6B1 shows total (t)-FOXO3a and NF-κB RelA in P72 and R72 macrophages treated as in FIG. 6A1 by immunofluorescence (DAPI-blue nuceli, scale bar-20 μm). FIGS. 6B2 and 6B3 show quantification of data based on FIG. 6B1. FIG. 6C1 show nuclear p-p53 from macrophages as in FIG. 6A1 by western blotting. FIG. 6C2 shows densitometry based on FIG. 6C1. FIG. 6C3 shows t-p53 in macrophages as in FIG. 6A1 by immunofluorescence (DAPI-blue nuclei, scale bar-20 μm). FIG. 6C4 shows quantification of data based on FIG. 6C3. FIGS. 6D1 and 6D2 show Nos2 and Socs1 expression in P72 and R72 macrophages treated as in FIG. 6A1. Bar graphs and horizontal lines in dot plots represent means±SEM. *p<0.05, **p<0.01, ***p<0.001 by t-test, and ****p<0.0001. Data are representative of two independent experiments.

FIGS. 7A1-7G shows the impact of mitochondrial R72 p53 on redox and PTEN. FIG. 7A1 shows plasma membrane PTEN and PIP3 in unstimulated and LPS-stimulated P72 and R72 macrophages by western blotting. FIGS. 7A2 and 7A3 show densitometry based on FIG. 7A1. FIG. 7B1 shows PTEN at non-reducing (oxidized/Oxi) and/or reducing (Red) conditions based on whole cell lysate (WCL) of macrophages treated as in FIG. 7A1 by western blotting. FIG. 7B2 shows densitometry of oxidized PTEN based on 7B1 at 30 min. FIG. 7C shows mitochondrial reactive oxygen species (ROS) by fluorescence assay. FIG. 7D shows mitochondrial membrane integrity by FACS-assessed Mito-Tracker accumulation. FIG. 7E1 shows p53 in mitochondria labelled by Mito-Tracker by immunofluorescence (DAPI-blue nuclei, arrows indicate colocalization, scale bar-30 μm). FIG. 7E2 shows mitochondrial p53 based on p53 & Mito Tracker colocalization as in FIG. 7E1. FIG. 7F shows p53 and MnSOD in p53 immunoprecipitates (IP) from mitochondrial fractions of unstimulated or LPS-stimulated macrophages by western blotting. FIG. 7G shows MnSOD activity in mitochondrial fractions. Bar graphs and horizontal lines represent means±SEM. *p<0.05; **p<0.01; ***p<0.001; and ****p<0.0001 by t-test or by Two-way ANOVA for G. Data are representative of two to three independent experiments.

FIGS. 8A-8G show P72 and R72 macrophages and tumor growth. FIG. 8A shows volume of tumors generated by subcutaneous injections of tumor cells (TC1), mixture of tumor cells and LPS-stimulated P72 macrophages (TC1+P72^{LPS}), or mixture of tumor cells and LPS-stimulated R72 macrophages (TC1+R72^{LPS}). FIG. 8B shows gating and identification of M1-like (CD11b⁺F4/80⁺ MHCII^{high}CD38^{high}CD206^{low}) and M2-like (CD11b⁺F4/80⁺ MHCII^{low}CD38^{low}CD206^{high}) macrophage subpopulations in tumors. FIG. 8C shows M1-like macrophage subpopulations in tumor types as in FIG. 8A. FIG. 8D shows M2-like macrophage subpopulations in tumor types as in FIG. 8A. FIG. 8E shows percentages of tumor CD4⁺ T cells out of CD3⁺ cells. FIG. 8F shows percentages of tumor CD8⁺ T cells out of CD3⁺ cells. FIG. 8G shows IFN-γ expression in spleen CD8⁺ T cells. Data are representative of one experiment with n=5-10 mice. *p<0.05; **p<0.01; ***p<0.001; and ****p<0.0001 by t-test and Two-Way ANOVA for FIG. 8A.

FIGS. 9A-9H show P72 and R72 mice response to LPS. FIG. 9A shows images of mice i.p. injected with LPS. FIG. 9B shows Kaplan-Meier analysis of the overall survival of mice challenged with LPS. FIG. 9C shows serum Creatinine, and BUN from mice shown in FIGS. 9A and 9B. FIG. 9D shows hematoxylin and eosin (H&E) staining of liver section from mice as in FIGS. 9A and 9B. FIG. 9E shows cytokines and chemokine response in mice as in FIGS. 9A and 9B. FIG. 9F shows experimental design for macrophage depletion and adoptive transfer. FIG. 9G1 shows representative FACS plot of mouse spleen macrophages. FIG. 9G2 shows quantification of FIG. 9G1. FIG. 9H shows Kaplan-Meier analysis of the overall survival of LPS-injected R72 mice transplanted with P72 and R72 BMDMs. Data represents mean±SEM (n=3-5, for FIGS. 9A, 9C, 9D, 9E, 9G1, and 9G2; n=10-11 for FIGS. 9B and 9H). Statistical analysis was performed using an unpaired t-test for FIGS. 9C, 9E and 9G2) and Gehan-Breslow-Wilcoxon test for FIGS. 9B and 9H). *p<0.05; ***p<0.001.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Current methods and systems for assessing disease prognosis have numerous limitations. For instance, many virus-mediated infections result in sepsis, which may also include a cytokine storm. The cytokine storm may in turn contribute to multiorgan failure and other life-threatening complications. However, the severity and onset of such cytokine storms in different individuals is difficult to predict. Similar difficulties exist in predicting the inflammatory response and prognosis of different individuals to different diseases.

As such, a need exists for improved systems and methods for assessing disease prognosis in different individuals. Numerous embodiments of the present disclosure aim to address the aforementioned need.

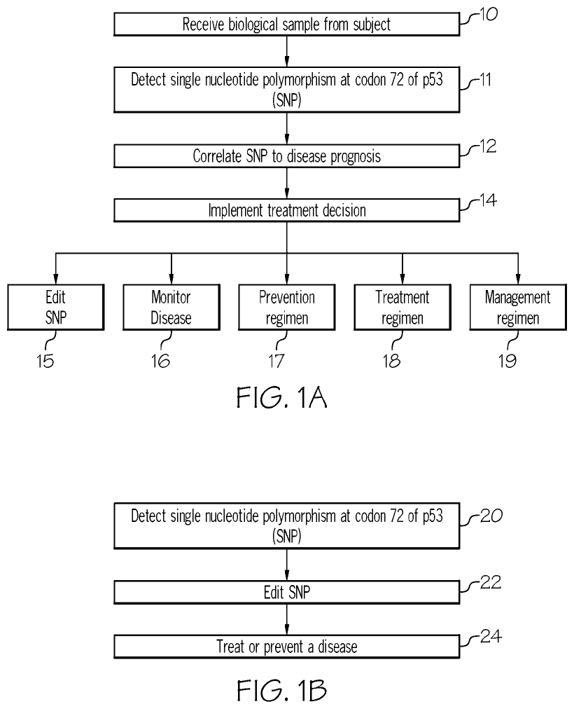

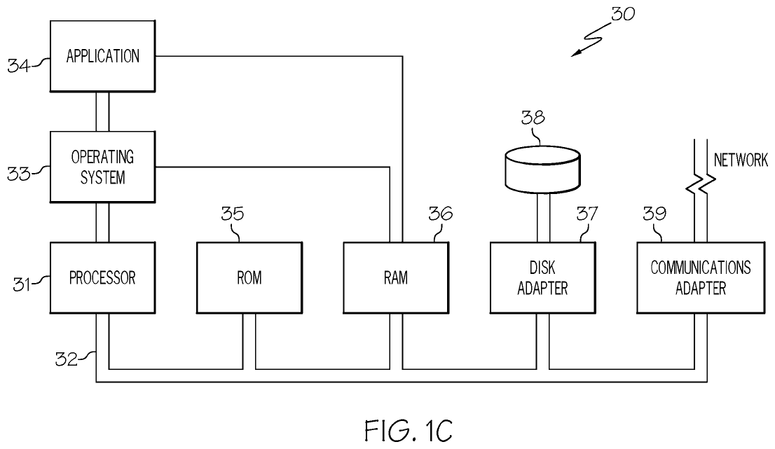

In some embodiments, the present disclosure pertains to a method of assessing a disease in a subject. In some embodiments illustrated in FIG. 1A, the methods of the present disclosure include: receiving a biological sample of the subject (step 10); detecting a single nucleotide polymorphism at codon 72 of the P53 protein (SNP) from the biological sample (step 11); and correlating the detected SNP to disease prognosis in the subject (step 12). In some embodiments further illustrated in FIG. 1A, the methods of the present disclosure also include a step of implementing a treatment decision based on the prognosis (step 14). For instance, in some embodiments, the treatment decision includes, without limitation, editing the detected SNP (step 15), monitoring the course of the disease (step 16), implementing a disease prevention regimen (step 17), implementing a disease treatment regimen (step 18), implementing a disease management regimen (step 19), or combinations thereof.

Further embodiments of the present disclosure pertain to methods of treating a subject. For instance, in some embodiments illustrated in FIG. 1B, the methods of the present disclosure include a step of detecting a single nucleotide polymorphism (SNP) at codon 72 of the P53 protein from a biological sample of the subject (step 20); and editing the detected SNP (step 22). In some embodiments further illustrated in FIG. 1B, the methods of the present disclosure may be utilized to treat or prevent a disease in a subject (step 24).

Additional embodiments of the present disclosure pertain to a diagnostic test for use in assessing a disease in a subject. In some embodiments, the diagnostic test includes: instructions for receiving at least one detected single nucleotide polymorphism at codon 72 of the P53 protein (SNP) from a biological sample of the subject; and instructions for correlating the detected SNP to disease prognosis in the subject. In some embodiments, the diagnostic test also includes instructions for implementing a treatment decision based on the prognosis.

Further embodiments of the present disclosure pertain to a computing device for assessing a disease in a subject. In some embodiments, the computing device includes one or more computer readable storage mediums having a program code embodied therewith. In some embodiments, the program code includes: programming instructions for receiving at least one detected single nucleotide polymorphism at codon 72 of the P53 protein (SNP) from a biological sample of the subject; and programming instructions for correlating the detected SNP to disease prognosis in the subject. In some embodiments, the computing devices of the present disclosure also include programming instructions for recommending a treatment decision based on the prognosis.

As set forth in more detail herein, the disease assessment methods, treatment methods, diagnostic tests, and computing devices of the present disclosure can include numerous embodiments.

SNP Detection from Biological Samples of Subjects

The disease assessment and treatment methods of the present disclosure can detect various SNPs from various biological samples of various subjects. Similarly, the diagnostic tests and computing devices of the present disclosure can receive various detected SNPs from various biological samples of various subjects. For instance, in some embodiments, the biological sample includes at least one of a tissue sample, a body fluid, a blood sample, or combinations thereof. In some embodiments, the disease assessment and treatment methods of the present disclosure also include a step of obtaining the biological sample from the subject.

Biological samples may be obtained from various subjects. Additionally, embodiments of the present disclosure may be utilized to assess, treat or prevent a disease in various subjects. For instance, in some embodiments, the subject is a human being. In some embodiments, the subject is a non-human mammal. In some embodiments, the non-human mammal includes, without limitation, a horse, a rabbit, a mouse, a rat, a pig, a sheep, a cow, a dog, or a cat. In some embodiments, the non-human mammal is a domestic animal, such as a dog or a cat. In some embodiments, the subject is suffering from a disease. In some embodiments, the subject is vulnerable to a disease.

Various SNPs may be detected from biological samples of subjects. For instance, in some embodiments, the detected SNP at codon 72 of the P53 protein is a codon expressing Proline (P72). In some embodiments, the detected SNP at codon 72 of the P53 protein is a codon expressing Arginine (R72). In some embodiments, the detected SNP is in homozygous form. In some embodiments, the detected SNP is in heterozygous form.

SNPs may be detected from biological samples of subjects in various manners. For instance, in some embodiments, SNP detection includes sequencing the SNP. In some embodiments, the detection of SNP occurs by a method that includes, without limitation, a polymerase chain reaction (PCR), next-generation sequencing (NGS), microarray-based sequencing, or combinations thereof.

### Assessment and Treatment of Diseases

The disease assessment methods, diagnostic tests and computing devices of the present disclosure can assess various diseases. Similarly, the treatment methods of the present disclosure can be utilized to treat or prevent various diseases. For instance, in some embodiments, the disease is associated with macrophage dysregulation. In some embodiments, the disease includes, without limitation, non-cancer related diseases, inflammatory-related diseases, cardiovascular diseases, hyper-inflammation, infections, sepsis, or combinations thereof.

In some embodiments, the disease includes an infection. In some embodiments, the infection includes, without limitation, a bacterial infection, a viral infection, a fungal infection, or combinations thereof. In some embodiments, the disease includes a viral infection, such as a SARS-COV-2 infection. In some embodiments, the disease includes sepsis. In some embodiments, the sepsis includes a virus-induced sepsis.

In some embodiments, the disease includes a cardiovascular disease. In some embodiments, the cardiovascular disease includes ischemic heart disease and stroke.

The disease assessment methods, diagnostic tests and computing devices of the present disclosure can assess diseases in various manners. For instance, in some embodiments, correlation of a detected SNP to disease prognosis includes correlating the detected SNP to disease susceptibility. In some embodiments, a detected codon expressing Proline (e.g., a detected C allele) at codon 72 of the P53 protein (P72) is correlated to a higher disease susceptibility relative to subjects without P72. In some embodiments, a detected codon expressing Arginine (e.g., a detected G allele) at codon 72 of the P53 protein (R72) is correlated to a lower disease susceptibility relative to subjects without R72.

In some embodiments, the correlation of a detected SNP to disease prognosis includes correlating the detected SNP to a subject's inflammatory response against the disease. In some embodiments, a detected codon expressing Proline (e.g., a detected C allele) at codon 72 of the P53 protein (P72) is correlated to a higher inflammatory response relative to subjects without P72. In some embodiments, a detected codon expressing Arginine (e.g., a detected G allele) at codon 72 of the P53 protein (R72) is correlated to a lower inflammatory response relative to subjects without R72.

In some embodiments, the correlation of a detected SNP to disease prognosis includes correlating the detected SNP to disease severity. In some embodiments, a detected codon expressing Proline (e.g., a detected C allele) at codon 72 of the P53 protein (P72) is correlated to a more severe disease relative to subjects without P72. In some embodiments, a detected codon expressing Arginine (e.g., a detected G allele) at codon 72 of the P53 protein (R72) is correlated to a less severe disease relative to subjects without R72.

In some embodiments, the correlation of a detected SNP to disease prognosis includes correlating the detected SNP to sepsis. In some embodiments, a detected codon expressing Proline (e.g., a detected C allele) at codon 72 of the P53 protein (P72) is correlated to a more severe sepsis and higher mortality relative to subjects without P72. In some embodiments, a detected codon expressing Arginine (e.g., a detected G allele) at codon 72 of the P53 protein (R72) is correlated to a less severe sepsis and lower mortality relative to subjects without R72.

In some embodiments, the correlation of a detected SNP to disease prognosis includes correlating the detected SNP to cardiovascular disease. In some embodiments, a detected codon expressing Proline (e.g., a detected C allele) at codon 72 of the P53 protein (P72) is correlated to a higher cardiovascular disease risk relative to subjects without P72. In some embodiments, a detected codon expressing Arginine (e.g., a detected G allele) at codon 72 of the P53 protein (R72) is correlated to a lower cardiovascular disease risk relative to subjects without R72.

In some embodiments, a detected codon expressing Proline (e.g., a detected C allele) at codon 72 of the P53 protein (P72) is correlated to a higher death risk due to unstable coronary artery disease relative to subjects without P72. In some embodiments, a detected codon expressing Proline (e.g., a detected C allele) at codon 72 of the P53 protein (P72) is correlated to a higher death risk due to stroke related to atherosclerosis complications relative to subjects without P72.

### Disease Assessment Modes

The disease assessment methods, diagnostic tests and computing devices of the present disclosure can assess diseases in various modes. For instance, in some embodiments, the disease assessment occurs manually.

In some embodiments, the disease assessment occurs automatically through the utilization of an algorithm. In some embodiments, the computing devices and diagnostic tests of the present disclosure include the algorithm. In some embodiments, the algorithm is an L1-regularized logistic regression algorithm. In some embodiments, the algorithm is a machine learning algorithm trained on the SNPs. In some embodiments, the machine learning algorithm includes supervised learning algorithms. In some embodiments, the supervised learning algorithms include nearest neighbor algorithms, naïve-Bayes algorithms, decision tree algorithms, linear regression algorithms, support vector machines, neural networks, convolutional neural networks, ensembles (e.g., random forests and gradient boosted decision trees), or combinations thereof.

Machine learning algorithms may be trained in various manners. For instance, in some embodiments, the training includes: (1) feeding a first set of detected SNP levels into a machine learning algorithm, where the first set of detected SNP levels are correlated to a disease prognosis; (2) feeding a second set of detected SNP levels into the machine learning algorithm, where the second set of detected SNP levels are not correlated to a disease prognosis; and (3) training the machine learning algorithm to assess disease prognosis in a subject by comparing the first set of detected SNP levels with the second set of SNP levels.

### Treatment Decisions

In some embodiments, the disease assessment methods of the present disclosure can also include a step of implementing a treatment decision based on the disease prognosis. Similarly, in some embodiments, the diagnostic tests of the present disclosure can include instructions for implementing a treatment decision based on the disease prognosis. In some embodiments, the computing devices of the present disclosure also include programming instructions for recommending a treatment decision based on the disease prognosis.

In some embodiments, the treatment decision includes, without limitation, editing the detected SNP, monitoring the course of a disease, implementing a disease prevention regimen, implementing a disease treatment regimen, implementing a disease management regimen, or combinations thereof. In some embodiments, the treatment decision includes implementing a disease treatment regimen. In some embodiments, the disease treatment regimen includes administering a therapeutic agent to a subject.

In some embodiments, the treatment decision includes editing the detected SNP. The disease assessment and treatment methods of the present disclosure can edit detected SNPs in various manners. Moreover, the diagnostic tests and computing devices of the present disclosure can recommend various modes of SNP editing.

In some embodiments, SNP editing of the P53 protein includes replacement of P72 with R72. In some embodiments, SNP editing of the P53 protein includes replacement of R72 with P72.

SNP editing can occur in various manners. For instance, in some embodiments, SNP editing occurs through the utilization of a gene editing system to correct the SNP. In some embodiments, the gene editing system includes a clustered regularly interspaced short palindromic repeats (CRISPR)/Cas nuclease (Cas) system (CRISPR/Cas system). In some embodiments, the CRISPR/Cas system includes at least one Cas nuclease and at least one guide RNA. In some embodiments, the Cas nuclease includes, without limitation, class 2 of Cas nucleases, Cas 9, Cas Φ, CasΦ2, Cpf1, or combinations thereof.

### Diagnostic Tests

The disease assessment methods of the present disclosure can have various uses and applications. For instance, in some embodiments, the disease assessment methods of the present disclosure can be used as a component of a diagnostic test of the present disclosure for assessing a disease in a subject.

The diagnostic tests of the present disclosure can be in various arrangements. For instance, in some embodiments, the diagnostic tests of the present disclosure may be in the form of a point of care test. In some embodiments, the diagnostic tests of the present disclosure may be in the form of a hand-held device. In some embodiments, the diagnostic tests of the present disclosure may be suitable for use as a predictive tool for patient stratification.

In some embodiments, the diagnostic tests of the present disclosure may be in the form of a kit with instructions. For instance, in some embodiments, the kit may include a biological sample collector (e.g., a collection tube) with detailed instructions for obtaining a biological sample from a subject (e.g., blood samples and/or swabs). The collected biological samples may then be sent to a designated testing center, where polymerase chain reaction (PCR) is employed to amplify the target DNA region containing the SNP. Subsequently, the amplified DNA may undergo digestion with a specific restriction enzyme that targets the SNP site. The resulting fragments may then be separated via gel electrophoresis, allowing for the determination of genotype based on the presence or absence of specific fragment sizes. The results may then be processed and returned (e.g., within 2 weeks).

The diagnostic tests of the present disclosure may have various components. For instance, in some embodiments, the diagnostic tests of the present disclosure may also include a computing device of the present disclosure.

### Computing Devices

The computing devices of the present disclosure can include various types of computer-readable storage mediums. For instance, in some embodiments, the computer-readable storage medium can be a tangible device that can retain and store instructions for use by an instruction execution device. In some embodiments, the computer-readable storage medium may include, without limitation, an electronic storage device, a magnetic storage device, an optical storage device, an electromagnetic storage device, a semiconductor storage device, or combinations thereof.

A non-exhaustive list of more specific examples of suitable computer-readable storage medium includes, without limitation, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, or combinations thereof.

A computer-readable storage medium, as used herein, is not to be construed as being transitory signals per se. Such transitory signals may be represented by radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer-readable program instructions for computing devices can be downloaded to respective computing/processing devices from a computer-readable storage medium or to an external computer or external storage device via a network, such as the Internet, a local area network (LAN), a wide area network (WAN) and/or a wireless network. In some embodiments, the network may include copper transmission cables, optical transmission fibers, wireless transmission, routers, firewalls, switches, gateway computers and/or edge servers. In some embodiments, a network adapter card or network interface in each computing/processing device receives computer-readable program instructions from the network and forwards the computer-readable program instructions for storage in a computer-readable storage medium within the respective computing/processing device.

In some embodiments, computer-readable program instructions for carrying out operations of the present disclosure may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object-oriented programming language such as Smalltalk, C++, or the like, and procedural programming languages, such as the “C” programming language or similar programming languages.

In some embodiments, the computer-readable program instructions may execute entirely on the user's computer, partly on the user's computer, as a stand-alone software package, partly on the user's computer and partly on a remote computer or entirely on the remote computer or server. In the latter scenario, the remote computer may be connected in some embodiments to the user's computer through any type of network, including a LAN or a WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry in order to perform aspects of the present disclosure.

Embodiments of the present disclosure for assessing a disease as discussed herein may be implemented using a computing device illustrated in FIG. 1C. Referring now to FIG. 1C, FIG. 1C illustrates an embodiment of the present disclosure of the hardware configuration of a computing device 30 which is representative of a hardware environment for practicing various embodiments of the present disclosure.

Computing device 30 has a processor 31 connected to various other components by computing device bus 32. An operating system 33 runs on processor 31 and provides control and coordinates the functions of the various components of FIG. 1C. An application 34 in accordance with the principles of the present disclosure runs in conjunction with operating system 33 and provides calls to operating system 33, where the calls implement the various functions or services to be performed by application 34. Application 34 may include, for example, a program for assessing a disease as discussed in the present disclosure, such as in connection with FIGS. 1A-1B, 2A-2U, 3A-3M, 4A1-4G, 5A-5F2, 6A1-6D2, 7A1-7G, 8A-8G, and 9A-9H.

Referring again to FIG. 1C, read-only memory (“ROM”) 35 is connected to computing device bus 32 and includes a basic input/output computing device (“BIOS”) that controls certain basic functions of computing device 30. Random access memory (“RAM”) 36 and disk adapter 37 are also connected to computing device bus 32. It should be noted that software components including operating system 33 and application 34 may be loaded into RAM 36, which may be computing device's 30 main memory for execution. Disk adapter 37 may be an integrated drive electronics (“IDE”) adapter that communicates with a disk unit 38 (e.g., a disk drive). It is noted that the program for assessing a disease, as discussed in the present disclosure, such as in connection with FIGS. 1A-1B, 2A-2U, 3A-3M, 4A1-4G, 5A-5F2, 6A1-6D2, 7A1-7G, 8A-8G, and 9A-9H may reside in disk unit 38 or in application 34.

Computing device 30 may further include a communications adapter 39 connected to computing device bus 32. Communications adapter 39 interconnects computing device bus 32 with an outside network (e.g., wide area network) to communicate with other devices.

Aspects of the present invention are described herein with reference to flowchart illustrations and/or block diagrams of methods, apparatus (systems), and systems according to embodiments of the invention. It will be understood that each block of the flowchart illustrations and/or block diagrams and combinations of blocks in the flowchart illustrations and/or block diagrams can be implemented by computer-readable program instructions.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of computing devices, methods, and computing devices according to various embodiments of the present disclosure. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based computing devices that perform the specified functions or acts or carry out combinations of special purpose hardware and computer instructions.

### ADDITIONAL EMBODIMENTS

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. TP53 Codon 72 Polymorphism Impacts Macrophage Activation Through Reactive Oxygen Species-Dependent Cell Signaling Alterations

The role of the most common TP53 single nucleotide polymorphism (SNP) at codon 72, which encodes for proline (P72) or arginine (R72), in the regulation of the immune system has not yet been thoroughly explored. In this Example, Applicant found that this SNP contributes to aggravated inflammatory response in COVID-19 patients resulting from biased macrophage activation. R72-P53 inhibits mitochondrial manganese superoxide dismutase, leading to impaired reactive oxygen species scavenging, oxidation of tensin homolog deleted on chromosome 10 (PTEN), and, consequently, its inhibition. Reduced PTEN activity causes constitutive activation of phosphatidylinositol 3-kinase/Akt (PI3K/Akt) pathway, which restricts proinflammatory (M1) and promotes antiinflammatory (M2) phenotypes through NF-κB and p53 inhibition. In contrast, PTEN-reduced PI3K/Akt activity, in P72 carrying cells, favors M1 phenotypes. Lipopolysaccharide (LPS)-stimulated R72 macrophages fail to reduce tumor growth in a mouse model of cancer, in contrast to P72 macrophages, which preserve M1 phenotype in vivo and reduce tumor growth by enhancing antitumor T cell responses, consistent with antitumor functions of M1 macrophages. In addition, P72 macrophages contributed to increased mortality in a mouse model of LPS-induced endotoxemia. Therefore, given the high frequency of P72 in African Americans, cell signaling alterations driven by codon 72 of TP53 SNP may potentially contribute to differences in clinical outcomes and health disparities in common diseases associated with dysregulated macrophage activation.

### Example 1.1. Background

Growing evidence for the role of tumor suppressor p53 in the regulation of the immune system has recently emerged. P53 impacts cell signaling involved in the regulation of immunity and in turn some of these signaling pathways enhance TP53 transcription. For example, p53 directly transactivates several genes essential for antiviral immune responses, including CC-chemokine ligand 2 (CCL2), interferon (IFN) regulatory factor 5 (IFR5) and 9 (IFR9), protein kinase RNA-activated (PKR), toll-like receptor 3 (TLR3), and interferon-stimulated gene 15 (ISG15), whereas TP53 transcription is induced by interferon α/β receptor signaling. P53 is also implicated in preventing autoimmunity, as p53 deficiency in macrophages enhances the production of proinflammatory cytokines, regulated by NF-κB, such as interleukin (IL)-1, IL-6, IL-12, and tumor necrosis factor (TNF) that are involved in the development of autoimmunity. Additional mechanisms of maintaining the immune tolerance include p53 controlled transcription of forkhead box P 3 (Foxp3) in T regulatory cells in mice.

There are scarce reports on the role of p53 in macrophage polarization, which is a process fundamentally important for the activation of immune responses to different pathogens, resolution of inflammation, and tissue repair. Macrophage polarization, often referred to as the activation of macrophages, is a continuum of evolving cellular phenotypes, which are triggered by different stimuli and correspond to different functions. Based on these phenotypic and functional characteristics, several macrophage polarization states were identified.

For example, M1 macrophages arise in the highly inflammatory microenvironment regulated by TLR and IFN signaling that is important for immunity to bacteria and intracellular pathogens. M1-like macrophages also contribute to antitumor immunity. However, in sepsis, they are a source of cytokines and chemokines that are hallmarks of dysregulated immune response to infection and contribute to sepsis mortality. M2 macrophages are linked to T helper 2 (T_{H}2) cell responses to helminths and to T_{H}2 responses in asthma and allergy. In the tumor microenvironment, the increase in the number of M2-like macrophages and reduction in M1-like macrophages is associated with impaired antitumor immunity.

Macrophages in different polarization states often coexist under different pathophysiological conditions and heterogeneity in macrophage phenotypes reflects a dynamic cellular response that changes with time and tissue environment. P53 activity increases when macrophages are activated, and this increase is associated with the subsequent downregulation of genes characteristic for M2 macrophages. Increased p53 activity triggered by nutlin-3a leads to the further downregulation of M2 genes. Accordingly, the increased expression of M2 genes was observed in macrophages from p53 deficient mice. These findings implicate p53 as a checkpoint of M2 macrophage polarization. In addition, p53 was also demonstrated to co-stimulate, in concert with NF-κB, the induction of proinflammatory IL-6 in human monocytes and macrophages.

Despite better understanding of p53 functions in immunity, the role of the most common p53 single nucleotide polymorphism (SNP) at codon 72 in the regulation of immune responses and macrophage polarization has not yet been thoroughly explored. The nucleotide sequences at codon 72 of p53, CCC or CGC, encode for proline (P72 encoded by C allele) or arginine (R72 encoded by G allele), respectively. Globally, the increase in the frequency of the R72 is proportional to increasing latitude and colder winters. The P72 is an ancestral variant and its frequency increases closer to the equator (Africa), suggesting that there may be selection for the P72 allele linked to high ultraviolet exposure or higher temperatures. P72 is more frequent in African Americans.

Several studies have demonstrated that the codon 72 of p53 SNP impacts p53 function. In response to cellular stress, the P72 variant is a better inducer of cell-cycle arrest whereas R72 is more efficacious in triggering apoptosis. However, the impact of codon 72 SNP on the cell cycle and apoptosis are tissue and context dependent. Several epidemiological studies suggest the associations of codon 72 polymorphism with a risk for lung, prostate, and breast carcinomas. However, genome-wide association studies (GWAS) have failed to link this SNP to an increased risk for specific cancer.

The potential cross talk of the codon 72 SNP with immunity was originally suggested by the increased interaction of the P72 variant with p65 RelA subunit of NF-κB and the aggravated response to lipopolysaccharide (LPS) challenge of Human p53 exon 4-9 knock-in (Hupki) mice carrying P72 compared to R72 Hupki mice. Importantly, the majority of P72 mice died as a result of septic shock, whereas R72 mice survived.

Since LPS is a potent inducer of the proinflammatory M1 phenotype in macrophages, which is associated with the secretion of proinflammatory cytokines that can contribute to sepsis-associated mortality, Applicant hypothesizes that the codon 72 of p53 SNP impacts severity and quality of inflammatory response through the regulation of macrophage polarization.

Consistent with this notion, Applicant observed the association of this SNP with biomarkers of inflammation in hospitalized COVID-19 patients. To study mechanisms by which this SNP influences inflammation, Applicant assessed responses of bone-marrow derived macrophages from mice and healthy donors, carrying the P72 or R72 variant, to LPS or IL-4 that are inducers of M1 or M2 macrophage polarization, respectively. To test if LPS ex vivo-stimulated P72 and R72 macrophages maintain their functionality in vivo, Applicant examined growth of tumors enriched in macrophages carrying P72 or R72 variants and the impact of this enrichment on antitumor immunity. Applicant further assessed the impact of the codon 72 SNP of p53 on inflammatory response and mortality in a mouse model of LPS-induced endotoxemia.

### Example 1.2. Biased Inflammatory Response in COVID-19 Patients

The term viral sepsis is currently accepted to describe the clinical manifestations of severe or critically ill COVID-19 patients, as they meet the diagnostic criteria for sepsis and septic shock according to the Sepsis-3 International Consensus. Like in other forms of sepsis, patients with COVID-19 may experience a cytokine storm that contributes to multiorgan failure and life-threatening COVID-19 complications. M1 macrophages are among the main cell types responsible for cytokine storm in the early stages of COVID-19. Interestingly, SARS-COV-2 prevents the transition of M1 to anti-inflammatory M2 in later stages of the disease.

Therefore, given the impact of codon 72 of TP53 SNP on survival in a mouse model of sepsis, Applicant investigated the association of this SNP with biomarkers of inflammation and clinicopathological variables of 115 symptomatic and hospitalized COVID-19 patients (Table 1).

TABLE 1
Basic demographic characteristics of COVID-19 patients.

| Parameter | Total (n = 115, 100%) | |
|---|---|---|
| Age (Means ± SD in years, total) | 64.75 ± 15.96 | |
| Proline (P/P) | 60.2 ± 12.82 | |
| Heterozygous (P/R) | 65.63 ± 17.14 | |
| Arginine (R/R) | 66.43 ± 16.06 | |
| Male gender (n, %) | 70 | (61%) |
| Proline (P/P) | 16 | (23%) |
| Heterozygous (P/R) | 30 | (43%) |
| Arginine (R/R) | 24 | (34%) |
| Female gender (n, %) | 45 | (39%) |
| Proline (P/P) | 9 | (20%) |
| Heterozygous (P/R) | 18 | (40%) |
| Arginine (R/R) | 18 | (40%) |
| BMI (Mean ± SD, total) | 31.88 ± 9.307 | |
| Proline (P/P) | 32.86 ± 10.54 | |
| Heterozygous (P/R) | 30.91 ± 8.920 | |
| Arginine (R/R) | 32.41 ± 9.094 | |
| Genotype | | |
| Proline (P/P) | 25 | (21%) |
| Heterozygous (P/R) | 49 | (43%) |
| Arginine (R/R) | 41 | (36%) |
| Ethnicity | | |
| African American | 8 | (7%) |
| Proline (P/P) | 6 | |
| Heterozygous (P/R) | 2 | |
| Arginine (R/R) | 0 | |
| Caucasian white | 80 | (69.5%) |
| Proline (P/P) | 13 | |
| Heterozygous (P/R) | 36 | |
| Arginine (R/R) | 31 | |
| Hispanic | 26 | (22.6%) |
| Proline (P/P) | 6 | |
| Heterozygous (P/R) | 11 | |
| Arginine (R/R) | 9 | |
| Other | 1 | (0.9%) |

These patients included 25 (21%) homozygous for C allele (P/P), 41 (36%) homozygous for G allele (R/R) and 49 (43%) heterozygous (P/R). Consistent with data on racial distribution of P72 and R72, 75% of African Americans were P/P homozygous in comparison to only 16% of American Caucasians.

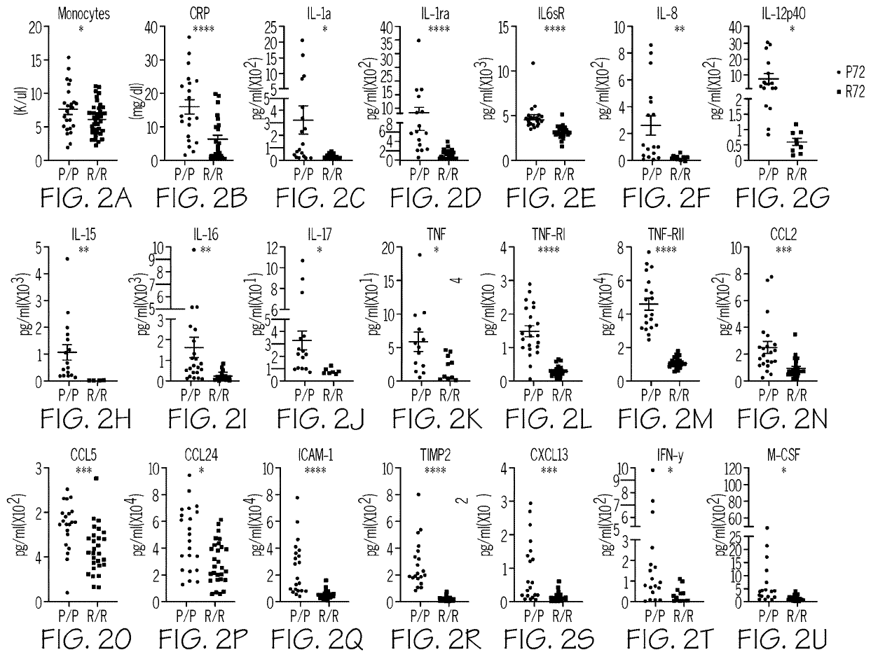

Total number of monocytes (FIG. 2A) and C-reactive protein (CRP) levels (FIG. 2B) were significantly higher in homozygous P/P vs. R/R patients. In addition, concentration of 19 out of 39 measured cytokines and chemokines were also significantly elevated in P/P vs. R/R patients (FIGS. 2C-2U), including cytokines implicated in early stage sepsis (TNF, IL-1a, IL-6sR, IL-8, and IFN-γ) and several secreted from human M1 macrophages (TNF, IL12 and CCL5). In addition, IFN-γ is a potent stimulator of M1 polarization.

Applicant did not find associations of codon 72 of p53 SNP with severity of infections or mortality, likely because only severely or critically ill patients with several comorbidities that influence clinical outcome were included in this Example. Thus, without being bound by theory, Applicant theorized that aggravated inflammatory response in P/P COVID-19 patients was associated with biased M1 macrophage polarization.

### Example 1.3. Biased Activation of Macrophages

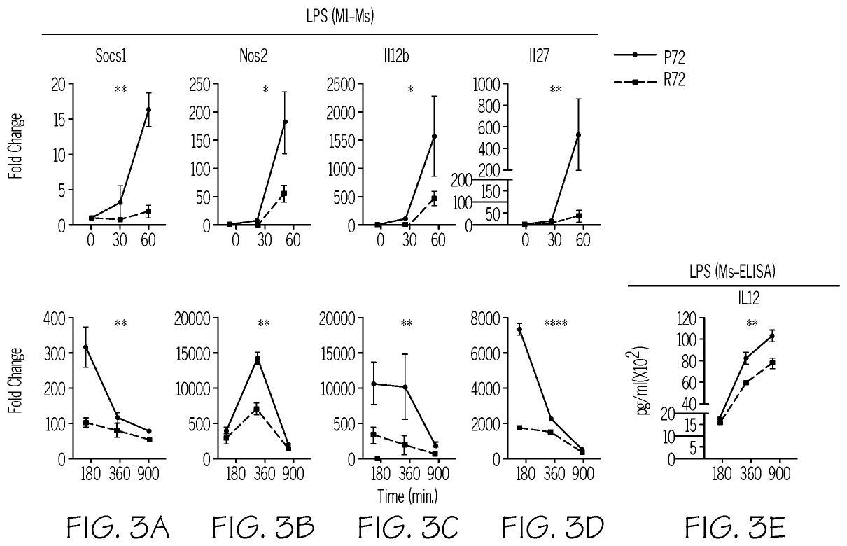

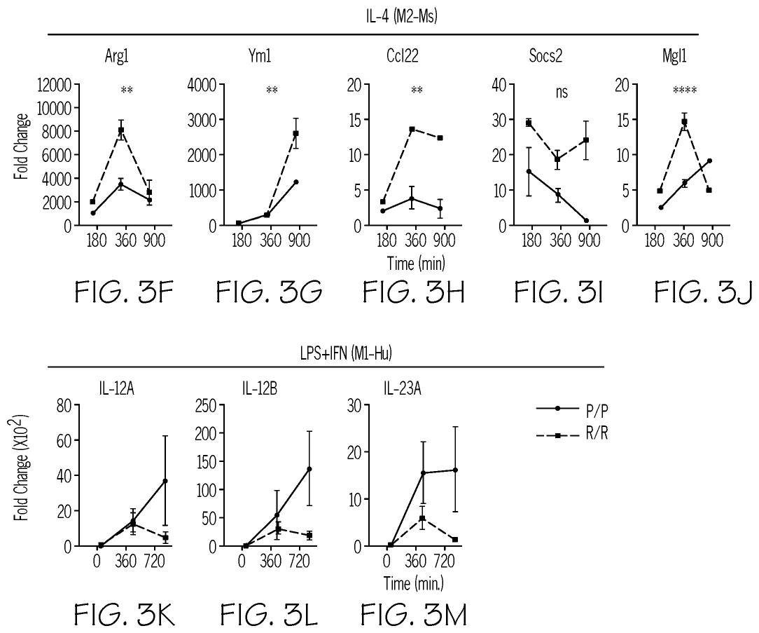

CSF-1 cultured macrophages from murine bone marrow remain the predominant and reproducible experimental in vitro system to study macrophage biology. Therefore, Applicant stimulated bone marrow-derived macrophages from Hupki P72 and R72 mice with LPS or IL-4, to generate M1 and M2 populations, respectively. LPS led to the greater upregulation of genes traditionally linked to M1 (LPS) phenotype, including Socs1, Nos2, Il12b and Il27, in P72 compared to R72 macrophages at early and late time points (FIGS. 3A-3D). In addition, P72 macrophages produced more IL-12 upon LPS stimulation than R72 cells, as Applicant found higher concentrations of this cytokine in the supernatant from LPS-stimulated P72 cells (FIG. 3E). In contrast, IL-4 stimulation (M2-inducer) led to greater upregulation of M2 genes, including Arg1, Ym1, Ccl22, Socs2, and Mgl1 in R72 vs. P72 macrophages, although only at late time points (FIGS. 3F-3J).

Similar to murine macrophages, P72 human blood mononuclear cells from healthy donors (Table 2) differentiated to macrophages and stimulated with LPS and IFN-γ (both M1-inducers), which showed a trend for greater upregulation of M1 genes when compared to R72 cells, although differences did not reach statistical significance (FIGS. 3K-3M). For human cells, Applicant used both LPS and IFN-γ, as these stimulants reflect better conditions during infection in vivo and, along with LPS, they are commonly used to induce M1 macrophage polarization. Thus, Applicant concluded that P72 macrophages are prone to become M1 polarized. In contrast, R72 variant of p53 reduces M1 and favors M2 phenotypes of macrophages upon activation.

TABLE 2
Demographic characteristics of healthy donors.

| S.N. | DIN | Age | Gender | Ethnicity | Genotype |
|---|---|---|---|---|---|
| 1 | W091022252703 | 39 | M | CA | GG |
| 2 | W091022252698 | 68 | M | CA | GG |
| 3 | W091022252700 | 76 | F | CA | CC |
| 4 | W091022407843 | 26 | M | CA | GG |
| 5 | W091022419376 | 50 | M | AA | CC |
| 6 | W091022410708 | 52 | M | AA | GG |
| 7 | W091023102997 | 35 | F | AA | CC |
| 8 | W091023126047 | 51 | M | AA | CC |
| 9 | W091023121085 | 63 | M | AA | CC |
| 10 | W091023126636 | 63 | M | AA | GG |
| 11 | W091023128652 | 51 | M | AA | CC |

### Example 1.4. Increased Activation of the PI3K/Akt Pathway and its Downstream Targets in R72 Macrophages

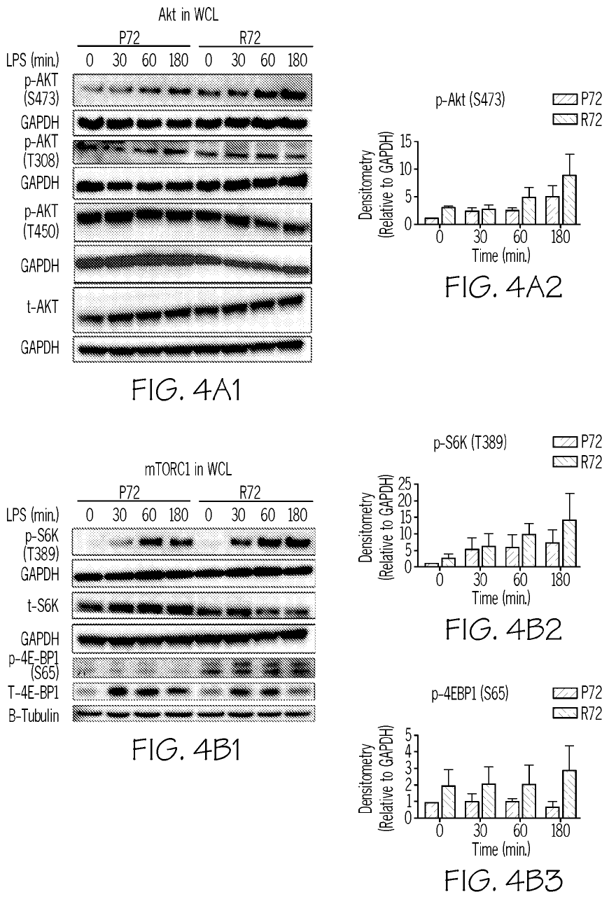

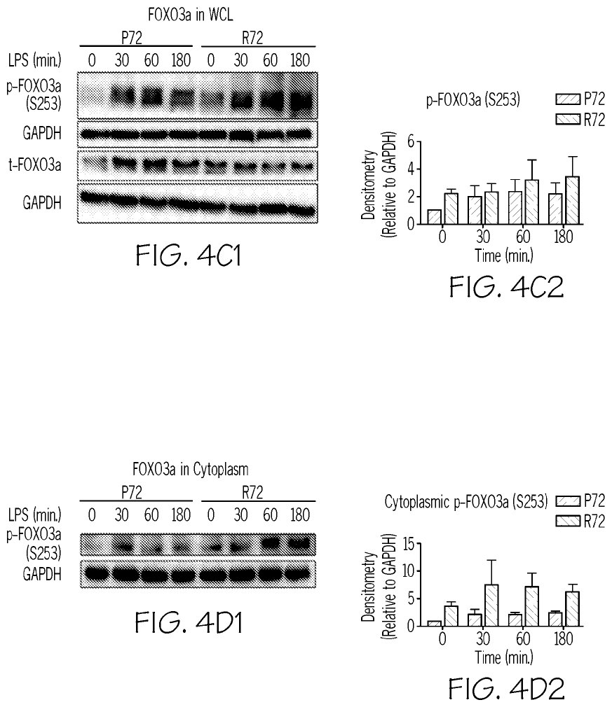

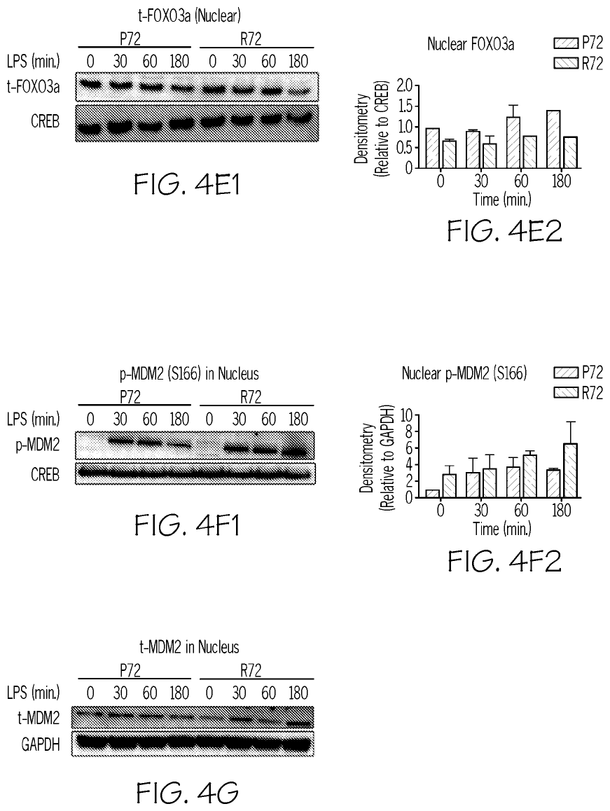

The phosphatidylinositol 3-kinase (PI3K)/Akt pathway and its downstream targets have emerged as key regulators of macrophage polarization. Activation of the PI3K/Akt pathway is essential for restricting proinflammatory (M1) and promoting anti-inflammatory (M2) responses in TLR4-stimulated macrophages. Since LPS stimulates TLR4 signaling and Applicant found that R72 macrophages expressed higher amounts of TLR4 than P72 cells (data not shown), Applicant examined PI3K/Akt activation in P72 and R72 macrophages upon LPS stimulation. The phosphorylation of Ser 473 residue of Akt was increased at baseline and increased further to a greater extent in R72 vs. P72 macrophages upon LPS treatment (FIGS. 4A1 and 4A2). No significant differences in the phosphorylation of Thr 308 or Thr 450 residues of Akt were observed (FIG. 4A1). The expression of total (t) Akt was not affected by codon 72 SNP either (FIG. 4A1).

This phosphorylation pattern suggests that Akt was phosphorylated by mechanistic target of rapamycin complex 2 (mTORC2). Activated Akt phosphorylates tuberin encoded by tuberous sclerosis complex (TSC) 2 and, therefore, inactivates TSC1/2 (tuberin and hamartin) complex. TSC1/2 inhibition leads to increased activation of mTORC1 via hyperactivation of Ras homolog enriched in brain (Rheb). Accordingly, Applicant found increased phosphorylation of mTORC1 downstream targets S6 kinase (S6K) (FIGS. 4B1 and 4B2) and eukaryotic translation initiation factor 4E-binding protein 1 (4E-BP1) (FIGS. 4B1 and 4B3) in R72 vs. P72 macrophages upon LPS stimulation, indicating Akt-dependent increase in mTORC1 activation. Total levels of both proteins were not affected by codon 72 of p53 SNP (FIG. 4B1).

An additional downstream target of Akt is transcription factor FOXO3a, which is a tumor suppressor and longevity factor. Consistent with increased Akt activity in R72 macrophages, the phosphorylation of FOXO3a at Ser 253 residue, based on whole cell lysate (WCL) western blotting, was greater in these cells than in P72 macrophages at the baseline and upon LPS stimulation (FIGS. 4C1 and 4C2), despite slight increase in total FOXO3a in P72 vs. R72 cells (FIG. 4C1). FOXO3a phosphorylation inhibits its transcriptional activity via a nucleus-to-cytoplasm shuttling mechanism. Accordingly, Applicant found more phosphorylated FOXO3a in the cytoplasm (FIGS. 4D1 and 4D2) and less total FOXO3a in nuclei of R72 vs. P72 cells (FIGS. 4E1 and 4E2).

Akt also phosphorylates and activates mouse double minute 2 homolog (Mdm2), which inhibits p53 by facilitating its ubiquitination and proteasomal degradation. p53 ubiquitination can occur in both the cytoplasm and the nucleus. However, ubiquitination inhibits p53 nuclear import, which is required for its transcriptional activity. In addition, Akt-mediated Mdm2 phosphorylation on Ser 166 and Ser 186 induces Mdm2 translocation to the nucleus. Expression of constitutively active Akt promotes nuclear entry of Mdm2, diminishes cellular levels of p53, and decreases p53 transcriptional activity. In agreement with these studies, Applicant found that increased Akt activity corresponded to the accumulation of phosphorylated at Ser 166 Mdm2 in nuclei of R72 cells upon LPS stimulation. In contrast, in P72 cells, the initial accumulation was followed by Mdm2 export from the nucleus (FIGS. 4F1 and 4F2). Levels of total Mdm2 in WCL were no significantly different (FIG. 4G).

### Example 1.5. Cytoplasmic NF-κB Restriction and Reduced p53 Nuclear Translocation in R72 Macrophages

NF-κB activation, nuclear translocation, and subsequent transcriptional regulation is key for the induction of several proinflammatory genes in M1 macrophages. Cytoplasmic FOXO3a was demonstrated to inhibit NF-κB via direct binding to NF-κB RelA in the cytoplasm and preventing its nuclear translocation in tumor associated dendritic cells. Therefore, Applicant theorized that similar mechanisms operate in R72 macrophages, as these cells have a larger pool of cytoplasmic phosphorylated FOXO3a (FIGS. 4D1-4D2).

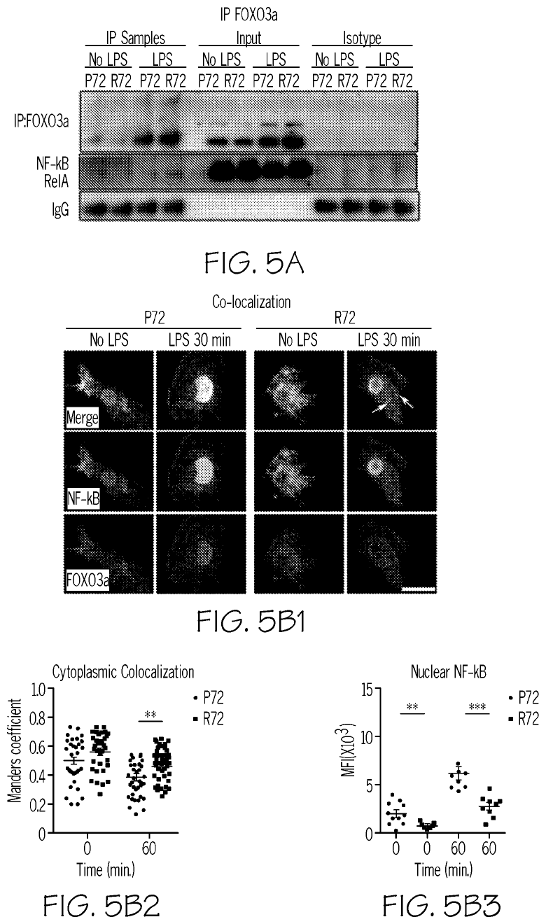

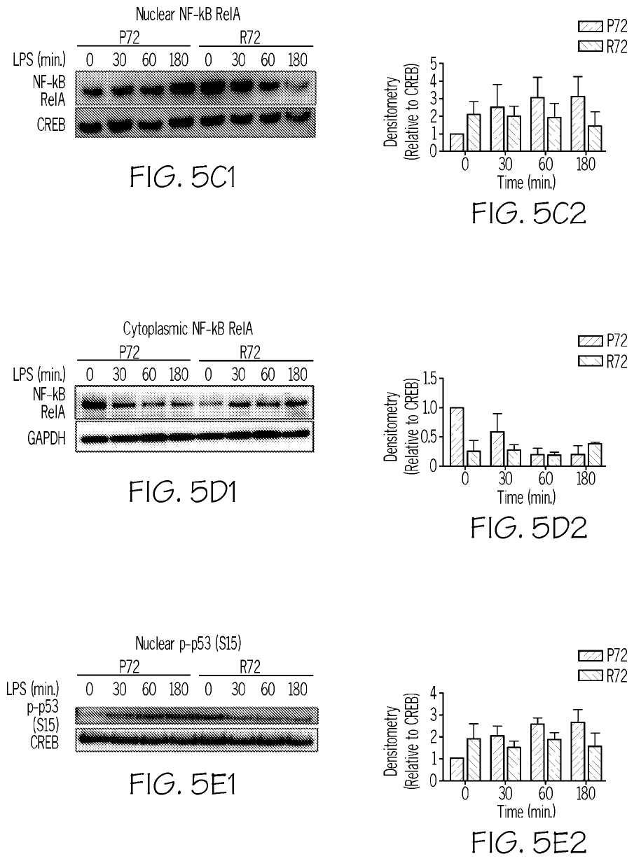

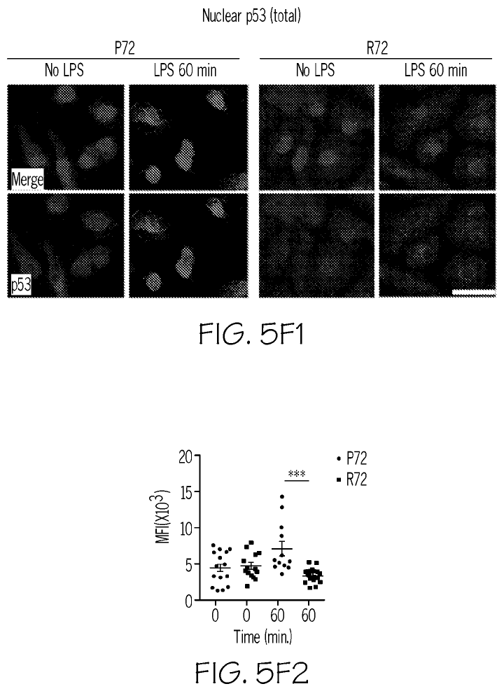

FOXO3a immunoprecipitation demonstrated the direct interaction of this protein with NF-κB RelA in the cytoplasm of R72 macrophages stimulated with LPS (FIG. 5A—ellipse). Confocal microscopy corroborated this data showing increased cytoplasmic colocalization of both proteins upon LPS stimulation (FIGS. 5B1 and 5B2) and impaired nuclear translocation of NF-κB in R72 vs. P72 cells (FIGS. 5B1 and 5B3). Applicant confirmed the impairment of NF-κB nuclear translocation by western blotting (FIGS. 5C1 and 5C2). In fact, the amount of RelA NF-κB in R72 cells was reduced upon LPS stimulation, in contrast to increases observed in P72 cells, although NF-κB was higher at the baseline in R72 compared to P72 macrophages, reflecting, perhaps, higher expression of TLR4 in R72 vs. P72 macrophages at the baseline (data not shown).

Increases in nuclear NF-κB RelA in P72 cells corresponded to reduction of cytoplasmic NF-κB RelA (FIGS. 5D1 and 5D2). Likewise, the reduction in nuclear NF-κB RelA in R72 corresponded to the increase in cytoplasmic fraction (FIGS. 5D1 and 5D2).

As anticipated, high amounts of phosphorylated Mdm2 in nuclei of R72 macrophages upon LPS stimulation (FIGS. 4F1-4F2) corresponded to lower amounts of phosphorylated p53 at Ser 15 residue and total p53 (FIGS. 5E1-5F2). The phosphorylation at Ser 15 is pivotal for p53 transcriptional activity. Thus, it is conceivable that the Akt-dependent impairment of nuclear translocation of NF-κB RelA and p53 both contribute to R72 bias toward M2 polarization, given the role of NF-κB and p53 crosstalk in the regulation of proinflammatory macrophage responses.

### Example 1.6. The Restoration of R72 Macrophages M1 Potential Upon Akt Inhibition

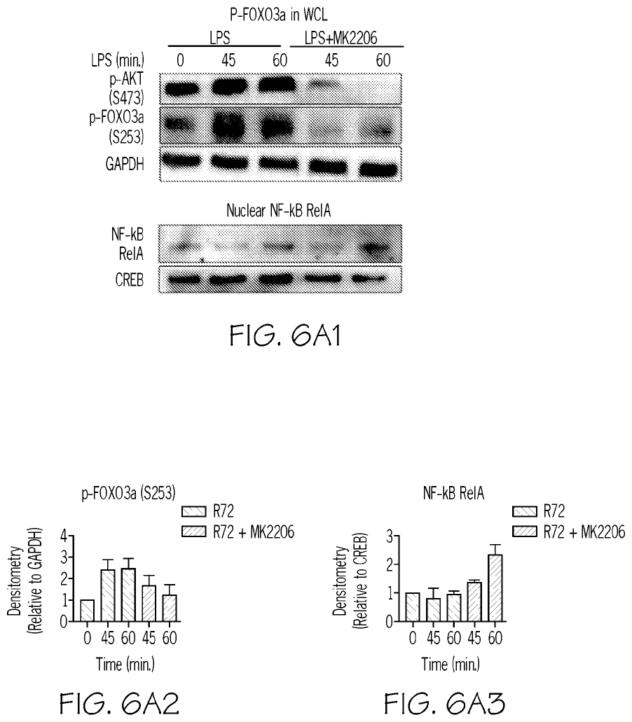

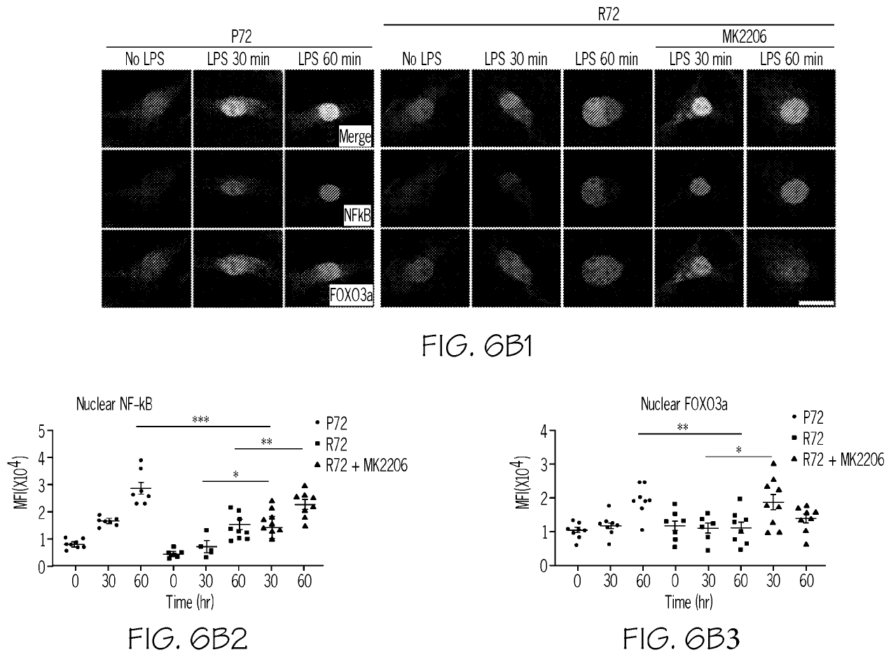

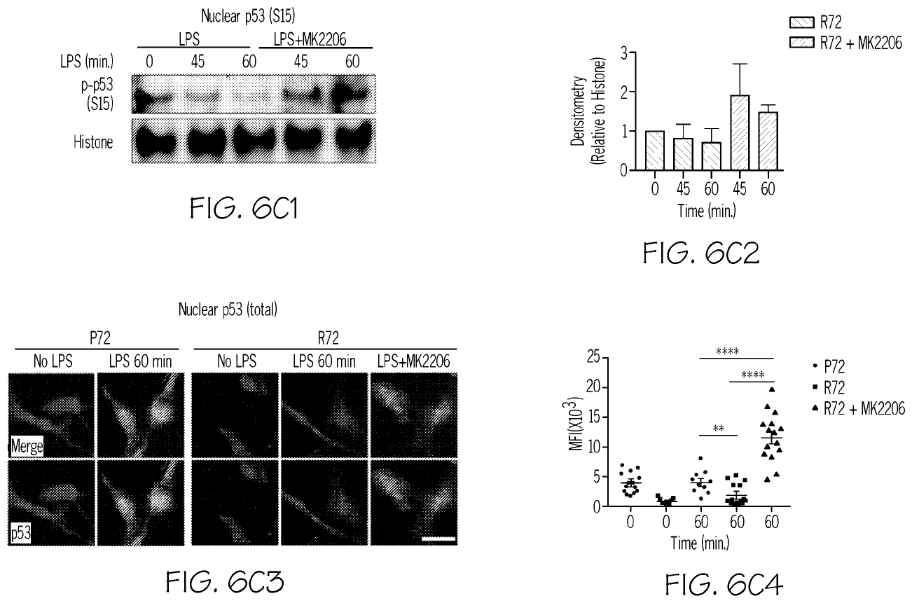

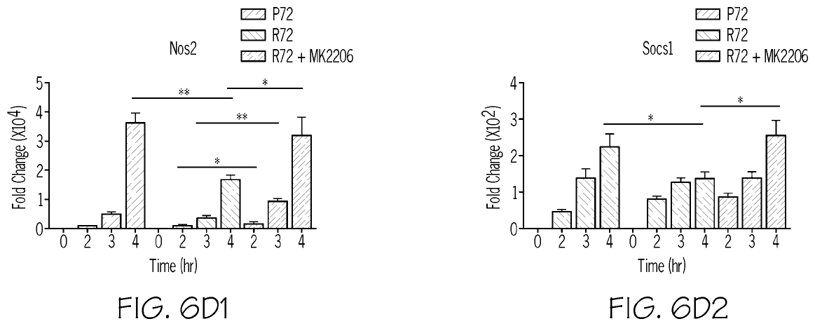

To support the key contributions of the PI3K/Akt signaling node in the biased response of R72 macrophages to TLR-4/LPS stimulation, Applicant blocked Akt activity in R72 macrophages with a highly selective Akt inhibitor MK-2206, which prevented Akt Ser 473 phosphorylation (FIG. 6A1). The reduced Akt phosphorylation resulted in reduced FOXO3a Ser 253 phosphorylation based on WCL and enhanced NF-κB RelA nuclear translocation at 60 minutes after LPS challenge (FIGS. 6A1, 6A2, and 6A3). Applicant corroborated these data by immunofluorescence and confocal microscopy (FIGS. 6B1, 6B2 and 6B3), which also demonstrated that Akt inhibition and reduced FOXO3a phosphorylation were associated with a greater amount of FOXO3a in nucleus (FIGS. 6B1 and 6B3), consistent with the role of this phosphorylation in FOXO3a nuclear export. In addition, Akt inhibition increased the amount of p53 phosphorylated at Ser 15 and total p53 in cell nuclei (FIGS. 6C1-6C4). These changes in Akt downstream signaling, resulting from Akt inhibition, increased expression of several M1 genes in R72 macrophages with the restoration of the expression of Nos2 and Socs1 to levels observed in P72 macrophages (FIGS. 6D1-6D2).

### Example 1.7. Reduced PTEN Activity and Constitutive PIK/Akt Activation as a Result of Oxidative Stress

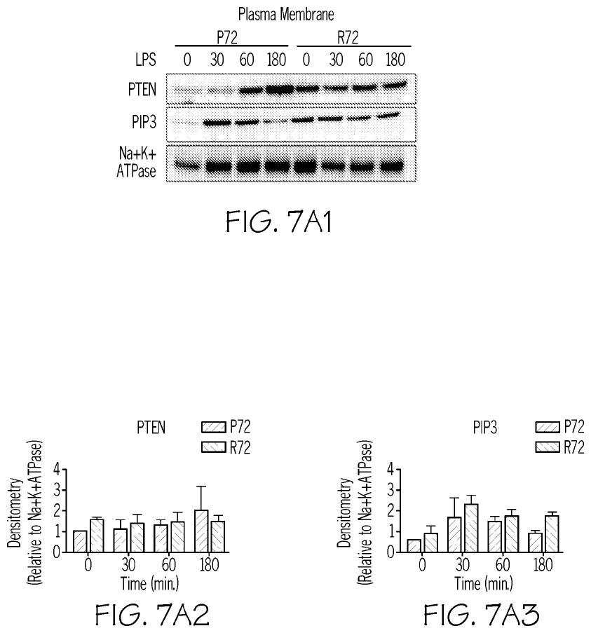

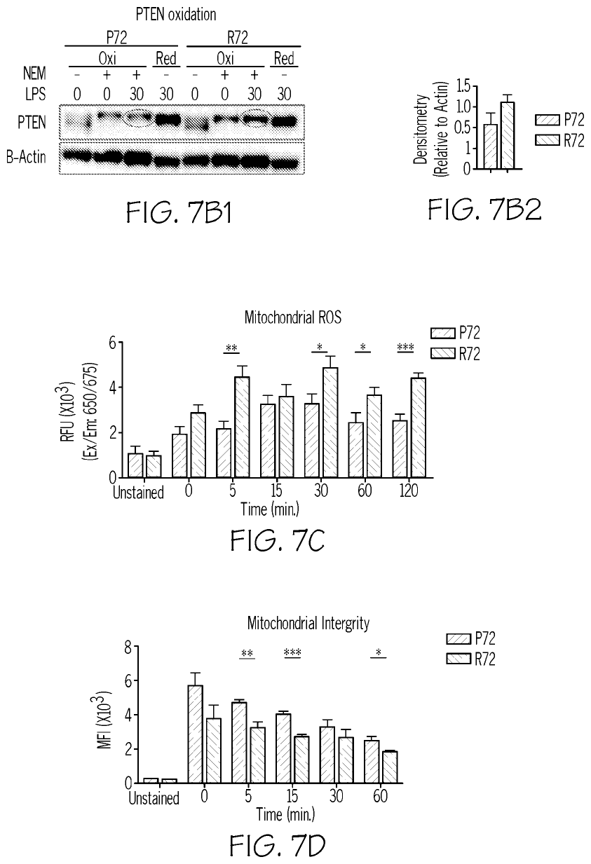

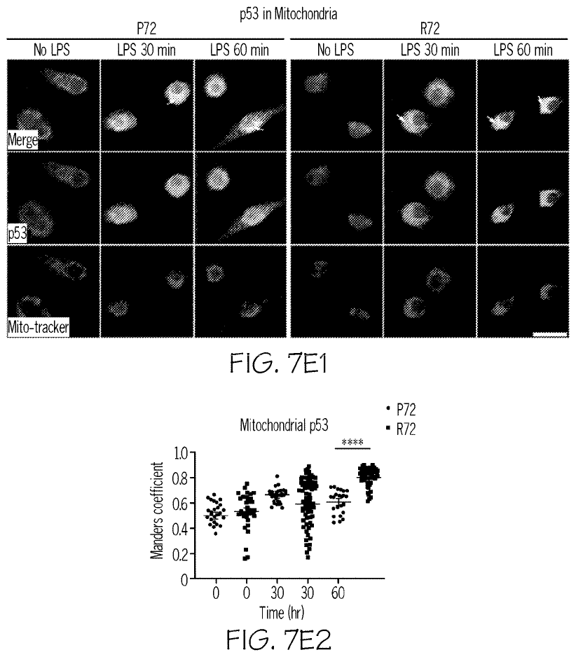

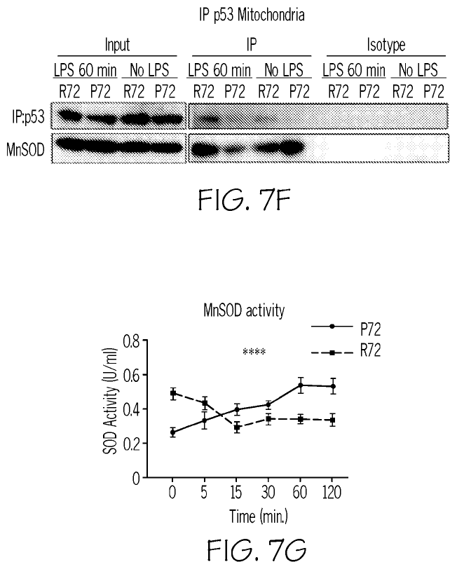

In response to extracellular cues, activated PI3Ks catalyze the formation of phosphatidylinositol triphosphate (PIP3) from PIP2 at the cell membrane. Akt is recruited to the cell membrane via binding to PIP3 and is activated following phosphorylation by PDK1 (at Thr 308) and by mTORC2 (at Ser 473). At the cell membrane, phosphatase and tensin homolog deleted on chromosome 10 (PTEN) dephosphorylates PIP3, thus, it acts as a main negative regulator of the PI3K/Akt pathway. Upon LPS activation, PTEN was recruited to, and accumulated, at the cell membrane in P72 cells (FIGS. 7A1 and 7A2). The cell membrane PIP3 amount in these cells initially increased upon LPS stimulation and then decreased (FIGS. 7A1 and 7A2) in parallel to increases in membrane PTEN. Thus, Applicant concluded that, in P72 cells, the initial PI3K/Akt pathway activation by LPS was tempered by the recruitment of active PTEN to the cell membrane. In R72 cells, PTEN was detected at the cell membrane prior to the stimulation with LPS, which did not cause further PTEN recruitment to the cell membrane (FIGS. 7A1 and 7A2). In contrast to P72 cells, PIP3 was enriched at the cell membrane prior to LPS stimulation and did not change as a result of this stimulation (FIGS. 7A1 and 7A2), apparently indicating reduced PTEN activity and, consequently, constitutive PI3K/Akt activation, supported by increased phosphorylation of Ser 473 Akt prior to LPS stimulation in R72 vs. P72 cells (FIGS. 4A1 and 4A2).

The oxidation of PTEN leads to its inactivation because it contains nucleophilic cysteine residues in the active site. This mechanism was likely responsible for PTEN inactivation in R72 cells, as they contained more oxidized PTEN than P72 cells prior to and 30 minutes after LPS stimulation (FIGS. 7B1 and 7B2). Higher amounts of oxidized PTEN corresponded to increased production of reactive oxygen species (ROS) in R72 cell mitochondria (FIG. 7C), which resulted in reduced mitochondrial membrane integrity (FIG. 7D). R72 p53 was reported to have enhanced mitochondrial localization resulting from nuclear export regulated by increased binding of phosphorylated MDM2 to R72 p53.

Applicant's data are consistent with the aforementioned observations, as Applicant found higher amounts of phosphorylated Mdm2 in nuclei of R72 macrophages (FIGS. 4F1 and 4F2), which was associated with the reduction of nuclear p53 (FIGS. 5E1-5F2). Accordingly, LPS stimulation resulted in greater accumulation of p53 in mitochondria of R72 than P72 macrophages (FIGS. 7E1 and 7E2).

In skin epidermal cells, mitochondrial p53 was reported to interact with manganese superoxide dismutase (MnSOD), which is the main superoxide scavenging enzyme, although direct impact on MnSOD activity was not clearly demonstrated. The immunoprecipitation of p53 confirmed this interaction in macrophages with stronger association of p53 with MnSOD2 in R72 cells (FIG. 7F). p53 inhibited MnSOD activity in R72 but not in P72 cells (FIG. 7G). Thus, Applicant concluded that PI3K/Akt activation, which drives biased macrophage R72 polarization, results from PTEN oxidation and inhibition caused by increased oxidative stress triggered by the inhibition of MnSOD by R72 p53.

### Example 1.8. LPS-Ex Vivo-Stimulated P72 and R72 Macrophages Maintain their Functionality in Mouse Model of Cancer and P72—but not R72 Macrophages Inhibit Tumor Growth

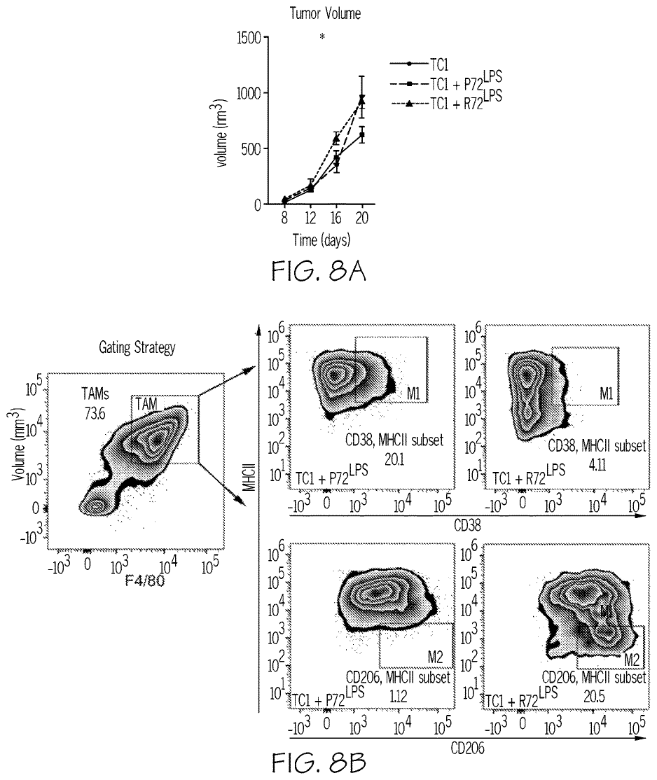

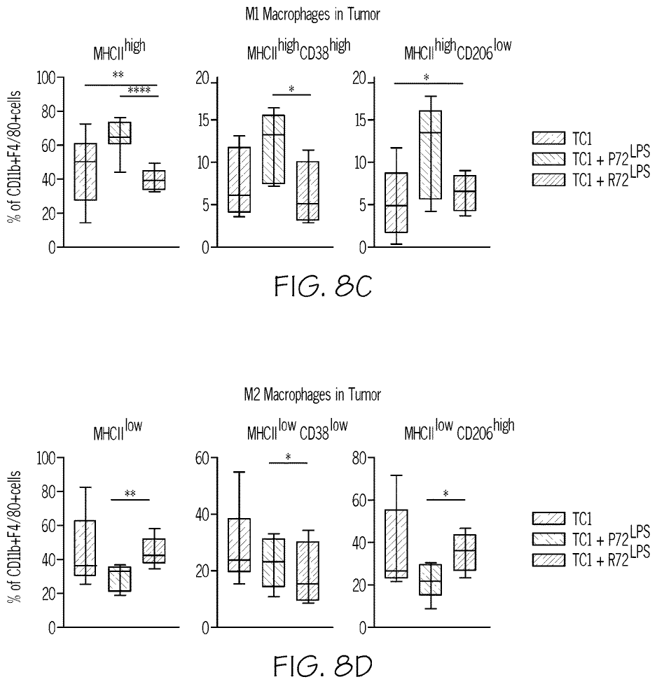

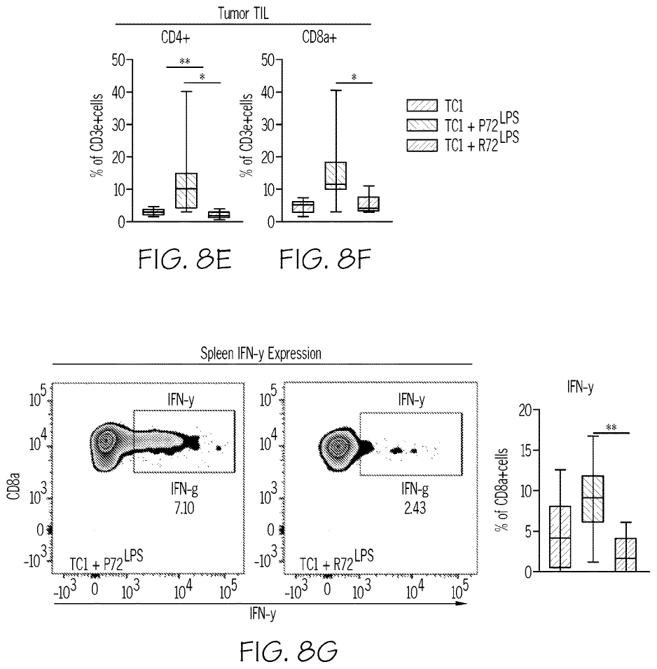

The majority of tumor associated macrophages (TAM) are M2-like in phenotype and these cells promote malignancy progression. However, the TAM population is heterogenous and it also contains M1-like macrophages that inhibit tumor growth by direct cytotoxicity and assisting in the development of efficacious antitumor T cell responses. Thus, Applicant theorizes that P72 macrophages induced to be M1-like macrophages have the potential to inhibit tumor growth, in contrast to R72 cells (M2-like). To test this, Applicant injected tumor cells (TC1) mixed with LPS stimulated P72 (TC1+P72^{LPS}) or R72 (TC1+R72^{LPS}) macrophages subcutaneously to mice. The addition of P72 macrophages (TC1+P72^{LPS}) reduced tumor growth in comparison to tumors generated by the injection of tumor cells alone (TC1), while the addition of R72 macrophages (TC1+R72^{LPS}) failed to produce such effect (FIG. 8A). The majority of TAM (F4/80⁺CD11b⁺) from TC1+P72^{LPS} tumors were MHCII^{high}CD38^{high}CD206^{low} cells corresponding to M1 macrophages (FIGS. 8B and 8C). In contrast, the majority of TAM from TC1+R72^{LPS} tumors were MHCII^{low}CD38^{low}CD206^{high} cells corresponding to M2 macrophages (FIGS. 8B and 8D). In addition, TC1+P72^{LPS} tumors were enriched with CD4⁺ and CD8⁺ T cells (FIGS. 8E and 8F). Additionally, splenic CD8⁺ T cells from mice bearing TC-1+P72^{LSP} tumor produced more IFN-γ (FIG. 8G), indicating better antitumor cytotoxic capacity of peripheral T cells.

### Example 1.9. P72 Macrophages Contributes to Increased Mortality in Mouse LPS-Induced Endotoxemia Model

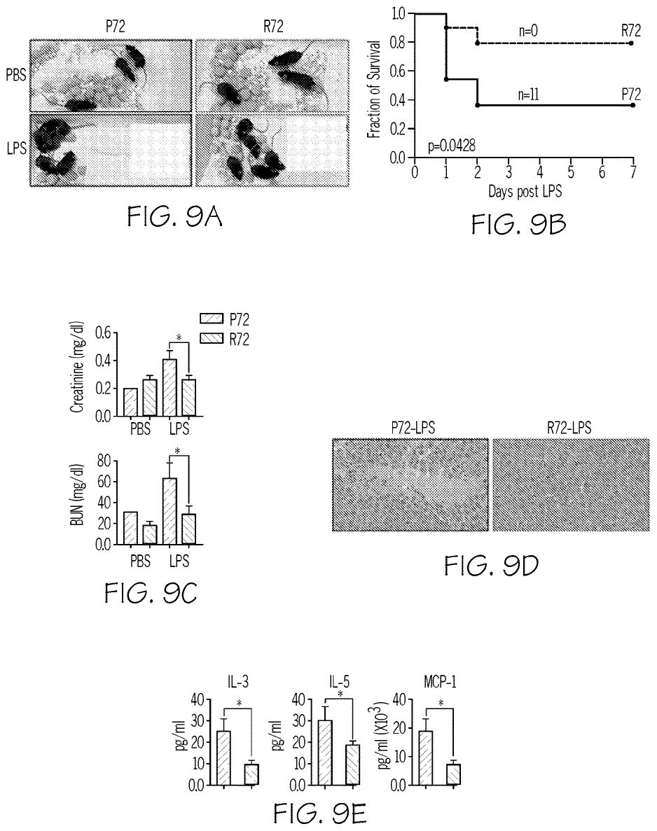

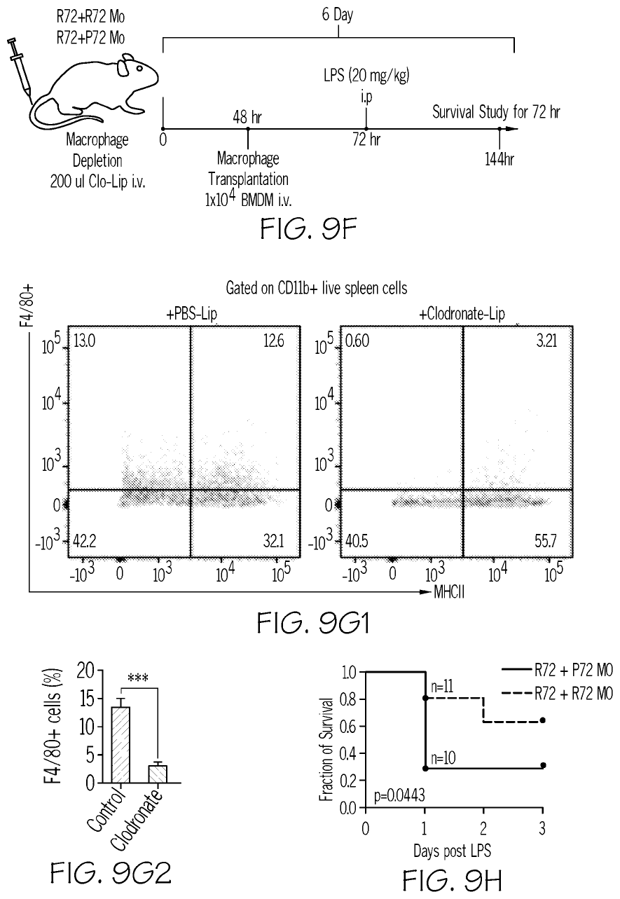

Applicant's findings that the codon 72 polymorphism influences the inflammatory response in COVID-19 patients prompted them to test the hypothesis that this polymorphism contributes to macrophage-dependent inflammatory responses and mortality in sepsis. To test this hypothesis, Hupki mice carrying P72 and R72 were intraperitoneally injected with LPS. LPS administration resulted in reduced motor activity and caused shivering (FIG. 9A) and greater weight loss in P72 mice (data not shown). The majority of P72 mice succumbed to septic shock within two days. In contrast, most of R72 mice survived (FIG. 9B). P72 mice exhibited higher levels of serum creatinine and blood urea nitrogen (BUN) when compared to R72 mice (FIG. 9C). Aspartate (AST) and alanine (ALT) transaminases, markers of liver damage, showed trend for grater increase in P72 mice without reaching statistical significance (data not shown). However, histology showed liver injury following LPS treatment only in P72 mice (FIG. 9D).

Excessive cytokine production in response to infection, driven by an immune response, is a cause of septic shock, multiorgan failure, and sepsis related mortality. Therefore, Applicant assessed cytokine and chemokine levels during endotoxemia. Notably, in P72 mice, there was a significant increase in IL-3 & IL-5 levels (FIG. 9E). Importantly, IL-3 deficiency has been demonstrated to confer protection against septic shock in mice, suggesting that IL-3 contributes to increased mortality in P72 mice following LPS exposure (FIG. 9B). In contrast, MCP-1 levels increased significantly in R72 (FIG. 9E), consistent with the protective role of MCP-1 against lethal endotoxemia in mice.

To confirm the pivotal role of P72 macrophages in LPS-induced mortality, Applicant depleted macrophages from R72 mice using clodronate liposomes and adoptively transferred these mice with BMDMs carrying P72 or R72 (FIGS. 9F and 9G1). The depletion of macrophages after clodronate administration was confirmed by analyzing F4/80⁺ macrophages in the spleen using fluorescence-activated cell sorting (FIGS. 9G1 and 9G2). The adoptive transfer of P72 BMDMs into R72 mice led to increase in mortality following LPS administration compared to R72 mice transferred with R72 BMDMs (FIG. 9H).

### Example 1.10. Discussion

Applicant's data provide evidence for the previously unknown link between quality and severity of the inflammatory response mediated by macrophages and the most common TP53 SNP at the codon 72. According to recent genomic sequencing data, the R72 variant of TP53 is present in 5.6 billion people worldwide, of which 2.3 billion are homozygous. The P72 is an ancestral variant and its frequency increases closer to the equator (e.g., Africa), suggesting that there may be selection for the P72 allele linked to high ultraviolet light exposure or higher temperatures. Up to 40% of Caucasian Americans are homozygous for R72, compared to only ~8% of African Americans, while P72 is more frequent in African Americans.

The biased inflammatory response driven by this SNP may have potential implications for several common diseases affecting millions of people worldwide, as dysregulated or biased macrophage activation contributes to pathogenesis of most common human pathologies. In early phases of sepsis, LPS and IFN-γ stimulated cells convert to M1 macrophages through TLR downstream signaling and secrete large quantities of proinflammatory cytokines that lead to exacerbated inflammation, progressive organ damage and, consequently, multiorgan failure.

Applicant's data that the biased inflammatory response to SARS-COV-2 infection is associated with codon 72 of TP53 SNP underscore clinical relevance of their findings. Data from mouse model of endotoxemia further support the possible role of codon 72 of p53 SNP in sepsis. In addition, M1 macrophages in the intima of arteries (known as foam cells) destabilize the atherosclerotic plaque and increase risk of cardiovascular complications including myocardial infarction. By contrast, Th2 cytokines that block M1 polarization have a protective role in atherosclerosis.

M2-like macrophages are associated with poor outcomes in cancer and several of their products suppress antitumor immunity. Therefore, Applicant's data from a mouse model of cancer suggest potential contributions of codon 72 of TP53 SNP to antitumor immunity.

M2 macrophages are also linked to Th2 responses in asthma and allergy and contribute to tissue fibrosis and macrophage activation contributes to the pathophysiology of diabetes mellitus. In fact, associations of codon 72 of p53 SNP with diabetes, obesity, and metabolic dysfunction have already been documented. A strong racial bias associated with codon 72 of TP53 SNP, demonstrated here signaling differences in human and murine macrophages imposed by this SNP, differences in inflammatory response to COVID-19 between P72 and R72 patients, and contributions of P72 macrophages to increased mortality in a model of endotoxemia suggest possible contributions of this SNP to health disparities, for example, increased sepsis-associated mortality in African Americans.

Applicant's mechanistic studies demonstrated that codon 72 of TP53 SNP drives biased activation of macrophages through its impact on redox homeostasis. R72 variant of p53 translocate to mitochondria upon TLR-4 stimulation, where it binds and inhibits MnSOD. P72 variant, in contrast, does not demonstrate this capacity. As a result, amounts of ROS in cells with R72 are increased leading to PTEN inhibition and PI3K/Akt activation, which skews macrophage polarization toward anti-inflammatory M2 phenotypes. The mutual links between p53 and redox homeostasis have been well described. P53 transcriptional activity is controlled by the redox status and p53 regulates redox homeostasis.

Early studies have demonstrated that binding of p53 to DNA requires reducing conditions. In contrast, oxidation inhibits p53. P53 transcriptional activity is regulated by several proteins implicated in redox homeostasis through redox-dependent and independent mechanisms. Importantly, p53 is responsible for transcriptional regulation of several genes that encode proteins with anti- and pro-oxidant properties. In addition, p53 interacts directly with proteins that have both anti- and pro-oxidant properties.

Not surprisingly, these interactions occur in mitochondria, where most of ROS is produced. P53 was found to interact with a key mitochondrial enzyme scavenging superoxide MnSOD, although impact of this interaction on MnSOD activity was not demonstrated. P53 increases stability of p66shc, which translocate to mitochondria, where it contributes to the generation of hydrogen peroxide. Despite this evidence, the impact of codon 72 of TP53 on redox homeostasis has not yet been reported. Thus, Applicant's data provides new insights into a pivotal role of p53 in redox homeostasis.

Further, Applicant found that cells carrying R72 have impaired mitochondrial membrane integrity likely as a result of increased ROS, which is consistent with a greater propensity of R72 to induce apoptosis. The connection that codon 72 of TP53 SNP with conserved PI3K/Akt pathway has an essential role in the regulation of metabolism, growth, proliferation, and cell survival provides evidence for the importance of this SNP for the regulation of basic cellular functions. As such, biased macrophage activation driven by codon 72 of TP53 SNP may contribute to differences in clinical outcomes and health disparities in most common human diseases including infection, diabetes mellitus, metabolic syndrome, obesity, cardiovascular diseases, and cancer.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of assessing a disease in a subject, said method comprising:
receiving a biological sample of the subject,
detecting a single nucleotide polymorphism at codon 72 of the P53 protein (SNP) from the biological sample; and
correlating the detected SNP to disease prognosis in the subject, wherein the disease is associated with macrophage dysregulation.

2. The method of claim 1, wherein the disease is selected from the group consisting of a non-cancer related disease, an inflammatory-related disease, a cardiovascular disease, hyper-inflammation, an infection, sepsis, virus-induced sepsis, a disease associated with macrophage dysregulation, or combinations thereof.

3. The method of claim 1, wherein the disease comprises an infection selected from the group consisting of a bacterial infection, a viral infection, a fungal infection, or combinations thereof.

4-5. (canceled)

6. The method of claim 1, wherein the correlation to disease prognosis comprises:
correlating the detected SNP to disease susceptibility, wherein a detected codon expressing Proline at codon 72 (P72) is correlated to a higher disease susceptibility relative to subjects without P72, and wherein a detected codon expressing Arginine at codon 72 (R72) is correlated to a lower disease susceptibility relative to subjects without R72;
correlating the detected SNP to disease severity, wherein a detected codon expressing Proline at codon 72 (P72) is correlated to a more severe disease relative to subjects without P72, and wherein a detected codon expressing Arginine at codon 72 (R72) is correlated to a less severe disease relative to subjects without R72;
correlating the detected SNP to the subject's inflammatory response against the disease, wherein a detected codon expressing Proline at codon 72 (P72) is correlated to a higher inflammatory response relative to subjects without P72, and wherein a detected codon expressing Arginine at codon 72 (R72) is correlated to a lower inflammatory response relative to subjects without R72;
correlating the detected SNP to sepsis, wherein a detected codon expressing Proline at codon 72 (P72) is correlated to a more severe sepsis and higher mortality relative to subjects without P72, and wherein a detected codon expressing Arginine at codon 72 (R72) is correlated to a less severe sepsis and lower mortality relative to subjects without R72;
correlating the detected SNP to cardiovascular disease, wherein a detected codon expressing Proline at codon 72 (P72) is correlated to a higher risk of cardiovascular disease relative to subjects without P72, and wherein a detected codon expressing Arginine at codon 72 (R72) is correlated to a lower risk of cardiovascular disease relative to subjects without R72; or
combinations thereof.

7-10. (canceled)

11. The method of claim 1, wherein the assessment occurs manually.

12. The method of claim 1, wherein the assessment occurs automatically through the utilization of an algorithm.

13. The method of claim 1, further comprising a step of implementing a treatment decision based on the prognosis.

14. The method of claim 13, wherein the treatment decision comprises editing the detected SNP, monitoring the course of the disease, implementing a disease prevention regimen, implementing a disease treatment regimen, implementing a disease management regimen, or combinations thereof.

15. The method of claim 13, wherein the treatment decision comprises editing the detected SNP, wherein the editing comprises:
replacement of a codon expressing Proline at codon 72 (P72) with a codon expressing Arginine at codon 72 (R72); or
replacement of a codon expressing Arginine at codon 72 (R72) with a codon expressing Proline at codon 72 (P72).

16-17. (canceled)

18. The method of claim 15, wherein the editing occurs through the utilization of a gene editing system to correct the SNP, wherein the gene editing system comprises a clustered regularly interspaced short palindromic repeats (CRISPR)/Cas nuclease (Cas) system (CRISPR/Cas system), wherein the CRISPR/Cas system comprises at least one Cas nuclease and at least one guide RNA.

19. (canceled)

20. The method of claim 13, wherein the treatment decision comprises implementing a disease treatment regimen, wherein the disease treatment regimen comprises administering a therapeutic agent to the subject.

21. The method of claim 1, wherein the subject is a human being.

22. (canceled)

23. A method of treating a subject, said method comprising:
detecting a single nucleotide polymorphism at codon 72 of the P53 protein (SNP) from a biological sample of the subject; and
editing the detected SNP.

24. The method of claim 23, wherein the method is used to treat or prevent a disease.

25. (canceled)

26. The method of claim 24, wherein the disease is selected from the group consisting of a non-cancer related disease, an inflammatory-related disease, a cardiovascular disease, hyper-inflammation, an infection, sepsis, virus-induced sepsis, a disease associated with macrophage dysregulation, or combinations thereof.

27. The method of claim 23, wherein the disease comprises an infection selected from the group consisting of a bacterial infection, a viral infection, a fungal infection, or combinations thereof.

28-29. (canceled)

30. The method of claim 23, wherein the editing comprises:
replacement of a codon expressing Proline at codon 72 (P72) with a codon expressing Arginine at codon 72 (R72); or
replacement of a codon expressing Arginine at codon 72 (R72) with a codon expressing Proline at codon 72 (P72).

31. (canceled)

32. The method of claim 23, wherein the editing occurs through the utilization of a gene editing system to correct the SNP, wherein the gene editing system comprises a clustered regularly interspaced short palindromic repeats (CRISPR)/Cas nuclease (Cas) system (CRISPR/Cas system), wherein the CRISPR/Cas system comprises at least one Cas nuclease and at least one guide RNA.

33. (canceled)

34. The method of claim 23, wherein the subject is a human being.

35. (canceled)

36. A diagnostic test for use in assessing a disease in a subject, wherein the diagnostic test comprises:
instructions for receiving at least one detected single nucleotide polymorphism at codon 72 of the P53 protein (SNP) from a biological sample of the subject; and
instructions for correlating the detected SNP to disease prognosis in the subject, wherein the disease is associated with macrophage dysregulation.

37. The method of claim 36, wherein the disease is selected from the group consisting of a non-cancer related disease, an inflammatory-related disease, a cardiovascular disease, hyper-inflammation, an infection, sepsis, virus-induced sepsis, a disease associated with macrophage dysregulation, or combinations thereof.

38. The diagnostic test of claim 36, wherein the disease comprises an infection selected from the group consisting of a bacterial infection, a viral infection, a fungal infection, or combinations thereof.

39-40. (canceled)

41. The diagnostic test of claim 36, wherein the subject is a human being.

42. (canceled)

43. The diagnostic test of claim 36, wherein instructions for correlating the detected SNP to disease prognosis comprises instructions for:
correlating the detected SNP to disease susceptibility, wherein a detected codon expressing Proline at codon 72 (P72) is correlated to a higher disease susceptibility relative to subjects without P72, and wherein a detected codon expressing Arginine at codon 72 (R72) is correlated to a lower disease susceptibility relative to subjects without R72;
correlating the detected SNP to disease severity, wherein a detected codon expressing Proline at codon 72 (P72) is correlated to a more severe disease relative to subjects without P72, and wherein a detected codon expressing Arginine at codon 72 (R72) is correlated to a less severe disease relative to subjects without R72;
correlating the detected SNP to the subject's inflammatory response against the disease, wherein a detected codon expressing Proline at codon 72 (P72) is correlated to a higher inflammatory response relative to subjects without P72, and wherein a detected codon expressing Arginine at codon 72 (R72) is correlated to a lower inflammatory response relative to subjects without R72;
correlating the detected SNP to sepsis, wherein a detected codon expressing Proline at codon 72 (P72) is correlated to a more severe sepsis and higher mortality relative to subjects without P72, and wherein a detected codon expressing Arginine at codon 72 (R72) is correlated to a less severe sepsis and lower mortality relative to subjects without R72;
correlating the detected SNP to cardiovascular disease, wherein a detected codon expressing Proline at codon 72 (P72) is correlated to a higher risk of cardiovascular disease relative to subjects without P72, and wherein a detected codon expressing Arginine at codon 72 (R72) is correlated to a lower risk of cardiovascular disease relative to subjects without R72; or
combinations thereof.

44-47. (canceled)

48. The diagnostic test of claim 36, further comprising instructions for implementing a treatment decision based on the prognosis.

49. The diagnostic test of claim 48, wherein the treatment decision comprises editing the detected SNP, monitoring the course of the disease, implementing a disease prevention regimen, implementing a disease treatment regimen, implementing a disease management regimen, or combinations thereof.

50. The diagnostic test of claim 48, wherein the treatment decision comprises editing the detected SNP, wherein the editing comprises:
replacement of a codon expressing Proline at codon 72 (P72) with a codon expressing Arginine at codon 72 (R72); or
replacement of a codon expressing Arginine at codon 72 (R72) with a codon expressing Proline at codon 72 (P72).

51-52. (canceled)

53. The diagnostic test of claim 48, wherein the treatment decision comprises implementing a disease treatment regimen, wherein the disease treatment regimen comprises administering a therapeutic agent to the subject.

54. A computing device for assessing a disease in a subject, wherein the computing device comprises one or more computer readable storage mediums having a program code embodied therewith, wherein the program code comprises:
programming instructions for receiving at least one detected single nucleotide polymorphism at codon 72 of the P53 protein (SNP) from a biological sample of the subject; and
programming instructions for correlating the detected SNP to disease prognosis in the subject, wherein the disease is associated with macrophage dysregulation.

55. The computing device of claim 54, wherein the disease is selected from the group consisting of a non-cancer related disease, an inflammatory-related disease, a cardiovascular disease, hyper-inflammation, an infections, sepsis, virus-induced sepsis, a disease associated with macrophage dysregulation, or combinations thereof.

56. The computing device of claim 54, wherein programming instructions for correlating the detected SNP to disease prognosis comprises programming instructions for:
correlating the detected SNP to disease susceptibility, wherein a detected codon expressing Proline at codon 72 (P72) is correlated to a higher disease susceptibility relative to subjects without P72, and wherein a detected codon expressing Arginine at codon 72 (R72) is correlated to a lower disease susceptibility relative to subjects without R72;
correlating the detected SNP to disease severity, wherein a detected codon expressing Proline at codon 72 (P72) is correlated to a more severe disease relative to subjects without P72, and wherein a detected codon expressing Arginine at codon 72 (R72) is correlated to a less severe disease relative to subjects without R72;
correlating the detected SNP to the subject's inflammatory response against the disease, wherein a detected codon expressing Proline at codon 72 (P72) is correlated to a higher inflammatory response relative to subjects without P72, and wherein a detected codon expressing Arginine at codon 72 (R72) is correlated to a lower inflammatory response relative to subjects without R72;
correlating the detected SNP to sepsis, wherein a detected codon expressing Proline at codon 72 (P72) is correlated to a more severe sepsis and higher mortality relative to subjects without P72, and wherein a detected codon expressing Arginine at codon 72 (R72) is correlated to a less severe sepsis and lower mortality relative to subjects without R72;
correlating the detected SNP to cardiovascular disease, wherein a detected codon expressing Proline at codon 72 (P72) is correlated to a higher risk of cardiovascular disease relative to subjects without P72, and wherein a detected codon expressing Arginine at codon 72 (R72) is correlated to a lower risk of cardiovascular disease relative to subjects without R72; or
combinations thereof.

57-60. (canceled)

61. The computing device of claim 54, further comprising programming instructions for recommending a treatment decision based on the prognosis.

62. The computing device of claim 61, wherein the treatment decision comprises editing the detected SNP, monitoring the course of the disease, implementing a disease prevention regimen, implementing a disease treatment regimen, implementing a disease management regimen, or combinations thereof.

63. The computing device of claim 61, wherein the treatment decision comprises editing the detected SNP, wherein the editing comprises replacement of a codon expressing Proline at codon 72 (P72) with a codon expressing Arginine at codon 72 (R72), or replacement of a codon expressing Arginine at codon 72 (R72) with a codon expressing Proline at codon 72 (P72).

64-65. (canceled)

66. The computing device of claim 61, wherein the treatment decision comprises implementing a disease treatment regimen, wherein the disease treatment regimen comprises administering a therapeutic agent to the subject.

67. The computing device of claim 54, wherein the computing device comprises an algorithm, wherein the algorithm is a machine learning algorithm trained on the SNP.

68. (canceled)

69. The computing device of claim 54, wherein the disease comprises an infection selected from the group consisting of a bacterial infection, a viral infection, a fungal infection, or combinations thereof.

70-71. (canceled)

72. The computing device of claim 54, wherein the subject is a human being.

73. (canceled)
